SEQUENCE LISTING (22 sequences)

SEQ ID NO: 1 - DNA, 97 bp, Homo sapiens
ataaaatttccaattggaacctaatgattcatcagactcagatatttaagttaacagtatttgagaatgatgaatcattaggttccggtcagaaatt

SEQ ID NO: 2 - DNA, 83 bp, Homo sapiens
agcttaggtaccaatttggccacaatgggttagaacactattccattgtgttcttacccaccatggccaaaattgggcctaag

SEQ ID NO: 3 - DNA, 85 bp, Homo sapiens
tgcaggcctctgtgtgatatgtttgatatattaggttgttatttaatccaactatatatcaaacatattcctacagtgtcttgcc

SEQ ID NO: 4 - DNA, 75 bp, Homo sapiens
tgccctggctcagttatcacagtgctgatgctgtctattctaaaggtacagtactgtgataactgaaggatggca

SEQ ID NO: 5 - DNA, 82 bp, Homo sapiens
gcttcgctcccctccgccttctcttcccggttcttcccggagtcgggaaaagctgggttgagagggcgaaaaaggatgaggt

SEQ ID NO: 6 - DNA, 83 bp, Homo sapiens
gtggtctcagaatcggggttttgagggcgagatgagtttatgttttatccaactggccctcaaagtcccgcttttggggtcat

SEQ ID NO: 7 - DNA, 110 bp, Homo sapiens
gtgataatgtagcgagattttctgttgtgcttgatctaaccatgtggttgcgaggtatgagtaaaacatggttccgtcaagcaccatggaacgtcacgcagctttctaca

SEQ ID NO: 8 - DNA, 90 bp, Homo sapiens
tttcctgccctcgaggagctcacagtctagtatgtctcatcccctactagactgaagctccttgaggacagggatggtcatactcacctc

SEQ ID NO: 9 - DNA, 82 bp, Homo sapiens
gcagtcctctgttagttttgcatagttgcactacaagaagaatgtagttgtgcaaatctatgcaaaactgatggtggcctgc

SEQ ID NO: 10 - DNA, 94 bp, Homo sapiens
gagtttggttttgtttgggtttgttctaggtatggtcccagggatcccagatcaaaccaggcccctgggcctatcctagaaccaacctaagctc

SEQ ID NO: 11 - DNA, 95 bp, Homo sapiens
ttgtacctggtgtgattataaagcaatgagactgattgtcatatgtcgtttgtgggatccgtctcagttactttatagccatacctggtatctta

SEQ ID NO: 12 - DNA, 84 bp, Homo sapiens
ccagtcacgtccccttatcacttttccagcccagctttgtgactgtaagtgttggacggagaactgataagggtaggtgattga

SEQ ID NO: 13 - DNA, 82 bp, Homo sapiens
agggggcgagggattggagagaaaggcagttcctgatggtcccctccccaggggctggctttcctctggtccttccctccca

SEQ ID NO: 14 - DNA, 86 bp, Homo sapiens
tgcttgtaactttccaaagaattctccttttgggctttctggttttattttaagcccaaaggtgaattttttgggaagtttgagct

SEQ ID NO: 15 - DNA, 110 bp, Homo sapiens
cgcctcagagccgcccgccgttcctttttcctatgcatatacttctttgaggatctggcctaaagaggtatagggcatgggaaaacggggcggtcgggtcctccccagcg

SEQ ID NO: 16 - DNA, 110 bp, Homo sapiens; identical to SEQ ID NO: 15

SEQ ID NO: 17 - DNA, 90 bp, Homo sapiens
tcccatgctgtgaccctctagaggaagcactttctgtttgttgtctgagaaaaaacaaagtgcttccctttagagtgttaccgtttggga

SEQ ID NO: 18 - DNA, 98 bp, Homo sapiens
gtgtagtagagctaggaggagagggtcctggagaagcgtggaccggtccgggtgggttccggcaggttctcaccctctctaggccccattctcctctg

SEQ ID NO: 19 - DNA, 78 bp, Homo sapiens
ctttctacacaggttgggatcggttgcaatgctgtgtttctgtatggtattgcacttgtcccggcctgttgagtttgg

SEQ ID NO: 20 - protein, 494 aa, Homo sapiens
MMYSPICLTQDEFHPFIEALLPHVRAIAYTWFNLQARKRKYFKKHEKRMSKDEERAVKDELLSEKPEIKQKWASRLLAKLRKDIRQEYREDFVLTVTGKKHPCCVLSNPDQKGKIRRIDCLRQADKVWRLDLVMVILFKGIPLESTDGERLMKSPHCTNPALCVQPHHITVSVKELDLFLAYYVQEQDSGQSGSPSHNDPAKNPPGYLEDSFVKSGVFNVSELVRVSRTPITQGTGVNFPIGEIPSQPYYHDMNSGVNLQRSLSSPPSSKRPKTISIDENMEPSPTGDFYPSPSSPAAGSRTWHERDQDMSSPTTMKKPEKPLFSSASPQDSSPRLSTFPQHHHPGIPGVAHSVISTRTPPPPSPLPFPTQAILPPAPSSYFSHPTIRYPPHLNPQDTLKNYVPSYDPSSPQTSQPNGSGQVVGKVPGHFTPVLAPSPHPSAVRPVTLSMTDTKPITTSTEAYTASGTSQANRYVGLSPRDPSFLHQQQSWYLG

SEQ ID NO: 21 - protein, 155 aa, Homo sapiens
MKVASGSTATAAAGPSCALKAGKTASGAGEVVRCLSEQSVAISRCAGGAGARLPALLDEQQVNVLLYDMNGCYSRLKELVPTLPQNRKVSKVEILQHVIDYIRDLQLELNSESEVGTPGGRGLPVRAPLSTLNGEISALTAEAACVPADDRILCR

SEQ ID NO: 22 - protein, 5 aa, Artificial Sequence
  Description of Artificial Sequence Synthetic peptide
VVVPP

(19) United States
(12) Patent Application Publication
(10) Pub. No.: US 2014/0155458 A1
(43) Pub. Date: Jun. 5, 2014

(54) Compositions and Methods for Treating Neoplasia

(71) Applicants: Nava Almog, Wayland, MA (US); Amir Abdollahi, Heidelberg (DE)

(72) Inventors: Nava Almog, Wayland, MA (US); Amir Abdollahi, Heidelberg (DE)

(73) Assignee: Genesys Research Institute, Brighton, MA (US)

(21) Appl. No.: 13/818,960

(22) PCT Filed: Aug. 24, 2011
(86) PCT No.: PCT/US2011/048951

§ 371 Date: Feb. 7, 2014

Related U.S. Application Data
(60) Provisional application No. 61/376,519, filed on Aug. 24, 2010.

(51) Int. Cl. C12N 15/113; C12Q 1/68

(52) U.S. Cl. CPC C12N 15/113; C12Q 1/6886
USPC 514/44 A; 435/375; 435/6.12

(57) ABSTRACT

The invention features compositions comprising microRNAs that are differentially regulated in dormant versus fast growing neoplasias, and related methods of using the microRNAs for inducing or prolonging dormancy in a neoplastic cell or otherwise inhibiting the growth of a neoplastic cell.

### CROSS-REFERENCE TO RELATED APPLICATION

This application claims the benefit of the following U.S. Provisional Application No. 61/376,519, filed Aug. 24, 2010, the entire contents of which are incorporated herein by reference.

### STATEMENT OF RIGHTS TO INVENTIONS MADE UNDER FEDERALLY SPONSORED RESEARCH

This work was supported by the following grants from NASA number NNJ06HA28G and U.S. Department of Energy number DE-SC0002606. The government has certain rights in the invention.

### BACKGROUND OF THE INVENTION

Cancer dormancy is a stage in tumor progression in which tumors remain occult and asymptomatic for a prolonged period of time. Cancer dormancy is an aspect of the earliest stages in tumor development, of micro-metastasis in distant organs, and of minimal residual disease left after surgical removal or treatment of primary tumors. Dormant tumors are usually at a size of a few millimeters in diameter, yet they can switch to become fast-growing, clinically-apparent, and potentially lethal tumors. Of clinical importance are the facts that dormant tumors are highly prevalent in the normal population and that dormant tumor cells left after primary tumor removal or treatment are commonly refractory to chemotherapy. Asymptomatic, small, and occult cancerous lesions are highly prevalent. The majority of these lesions never progress into the stage of exponential tumor growth. This implies that dormant tumors rarely succeed in overcoming inherent defense mechanisms against tumor development. Defense mechanisms implicated in limiting tumor development include tumor cell senescence, immune response of the host, hormonal control or block of tumor growth, and/or insufficiency of tumor angiogenesis potential. Moreover, delayed recurrences, common in breast cancer and other tumor types, are well explained by the concept of tumor dormancy. Therefore, cancer can remain occult and asymptomatic for years and decades while certain molecular and cellular mechanisms either halt or are insufficient to enable tumor progression and its mass expansion.

Tumor dormancy is a more common feature of cancer than appreciated and has significant implications in the prevention and treatment of cancer. Proteins differentially regulated in dormant tumors, relative to actively growing tumors, are likely to be useful as therapeutics or as therapeutic targets.

There is a need for agents and methods that will induce tumor dormancy, prolong dormancy, or otherwise inhibit the progression of dormant tumors to fast-growing tumors.

### SUMMARY OF THE INVENTION

As described below, the present invention features compositions and methods for inducing or prolonging dormancy in a neoplastic cell or otherwise inhibiting the growth of neoplastic cells.

The invention provides compositions and methods featuring microRNAs whose expression is increased in dormant tumors, and methods for inducing or prolonging tumor dormancy by increasing the expression of the microRNA in the tumor. Compositions and articles defined by the invention were isolated or otherwise manufactured in connection with the examples provided below. Other features and advantages of the invention will be apparent from the detailed description, and from the claims.

In one aspect, the invention generally features a method of inhibiting neoplastic cell growth, involving contacting a neoplastic cell (e.g., in vitro or in vivo) with a dormancy associated microRNA.

In another aspect, the invention features a method of inhibiting tumor progression, involving contacting a tumor with an effective amount of a dormancy associated microRNA.

In another aspect, the invention features a method of prolonging dormancy in a tumor, involving contacting the tumor with an effective amount of a dormancy associated microRNA.

In yet another aspect, the invention features a method of inhibiting angiogenesis in a tumor, involving administering to the subject (e.g., human) an effective amount of a dormancy associated microRNA.

In another aspect, the invention features a method of ameliorating a neoplasia in a subject, involving administering to the subject an effective amount of a dormancy associated microRNA.

In another aspect, the invention features a kit for the treatment of a neoplasia, the kit containing an effective amount of a dormancy associated microRNA and directions for using the kit for the treatment of a neoplasia.

In another aspect, the invention features a pharmaceutical composition for the treatment of a neoplasia containing an effective amount of a dormancy associated microRNA and a pharmaceutically acceptable excipient. In one embodiment, the composition further contains one or more chemotherapeutics (e.g., abiraterone acetate, altretamine, anhydrovinblastine, auristatin, azacitidin, bendamustin, bevacizumab, bexarotene, bicalutamide, BMS 184476, 2,3,4,5,6-pentafluoro-N-(3-fluoro-4-methoxyphenyl)benzene sulfonamide, bleomycin, bortezomib, N,N-dimethyl-L-valyl-L-valyl-N-methyl-L-valyl-L-proly-1-Lproline-t-butylamide, cachectin, capecitabin, cemadotin, cetuximab, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, dasatinib, daunorubicin, dolastatin, doxorubicin (adriamycin), erlotinib, etoposide, 5-fluorouracil, finasteride, flutamide, hydroxyurea and hydroxyureataxanes, ifosfamide, imatinib, irinotecan, lenalidomid, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, panitumumab, pazopanib, prednimustine, procarbazine, rituximab, RPR109881, sorafinib, stramustine phosphate, sunitinib, tamoxifen, tasonermin, taxol, temozolomide, transtuzumab, tretinoin, vinblastine, vincristine, vindesine sulfate, vinflunine, and vorinostat).

In another aspect, the invention features a method of characterizing the aggressiveness of a neoplasia, involving determining the level of expression of one or more dormancy associated microRNAs in a subject sample, where a decreased level of expression relative to a reference indicates that the neoplasia is aggressive whereas an increased level of expression relative to a reference indicates that the neoplasia is dormant.

In another aspect, the invention features a method of characterizing the aggressiveness of a neoplasia, involving determining the level of expression of one or more aggressiveness associated microRNAs in a subject sample, where an increased level of expression relative to a reference indicates that the neoplasia is aggressive, whereas a decreased level of expression relative to a reference indicates that the neoplasia is dormant.

In another aspect, the invention features a method of monitoring a subject diagnosed as having a neoplasia, the method involving determining the level of expression of one or more dormancy associated microRNAs in a subject sample, where an alteration in the level of expression relative to the level of expression in a reference indicates the severity of neoplasia in a subject.

In another aspect, the invention features a method of monitoring a subject being treated for a neoplasia, the method involving determining the level of expression of one or more aggressiveness associated microRNAs in a subject sample, where an alteration in the level of expression relative to the level of expression in a reference indicates the efficacy of the treatment in the subject.

In another aspect, the invention features a method of selecting a treatment regimen for a subject diagnosed as having a neoplasia, the method involving determining the level of expression of one or more dormancy-associated or aggressive-associated microRNAs in a subject sample relative to a reference, where the level of expression of the microRNA indicates an appropriate treatment regimen for the subject.

In another aspect, the invention features a diagnostic kit for the diagnosis of a neoplasia in a subject involving a nucleic acid probe capable of detecting a dormancy-associated microRNA and written instructions for use of the kit for detection of a neoplasia.

In another aspect, the invention features a method of altering the expression of a dormancy-associated microRNA in a cell, the method involving contacting the cell with an effective amount of an agent capable of altering the expression of the dormancy-associated microRNA.

In another aspect, the invention features a method of identifying a compound that inhibits a neoplasia, the method involving contacting a cell that expresses a dormancy-associated microRNA with a candidate agent, and comparing the level of expression of the microRNA in the cell with the level present in a control cell not contacted by the agent, where an increase in expression of the dormancy-associated microRNA identifies the agent as inhibiting a neoplasia.

In another aspect, the invention features a method of identifying an agent that inhibits a neoplasia, the method involving contacting a cell that expresses an aggressiveness-associated microRNA with a candidate compound, and comparing the level of expression of the microRNA in the cell with the level present in a control cell not contacted by the agent, where reduced expression of the aggressiveness-associated microRNA identifies the agent as inhibiting a neoplasia.

In another aspect, the invention features a method of identifying a candidate agent that inhibits a neoplasia, the method involving: contacting a cell containing a reporter molecule under control of a dormancy-associated microRNA promoter with a candidate compound; detecting the level of the reporter molecule expressed in the cell contacted with the candidate agent; and comparing the level of the reporter molecule expressed in the cell contacted with the candidate compound with the level of the reporter molecule expressed in a control cell not contacted with the candidate compound, where an alteration in the level of the reporter molecule expression identifies the candidate compound as a agent that inhibits neoplasia.

In another aspect, the invention provides a method of inhibiting neoplastic cell growth, involving contacting a neoplastic cell with an agent that inhibits the expression or activity of a protein or nucleic acid molecule that is down-regulated in response to Dmir overexpression in a neoplastic cell. In one embodiment, the protein is NFIB, ID1 or Tax3b, or a polynucleotide encoding said protein. In another embodiment, the agent is an inhibitory nucleic acid molecule, antibody, or small compound.

In various embodiments of any of the above aspects or any other aspect of the invention delineated herein, the dormancy associated microRNA is any one or more of (e.g., 2, 3, 4, 5, 6, 7, 8, 9, 10, 11, 12, 13, 14, 15, 16) has-miR-101, has-miR-320, has-miR-193b, has-miR-218, has-miR-151, has-miR-19a, has-miR-331, has-miR-340, has-miR-184, has-miR-186, DmiR3, has-miR-185, DmiR1, DmiR2, has-miR-202, and has-miR-545. In other embodiments, the dormancy associated microRNA is DmiR1, DmiR2, or DmiR3. In additional embodiments, the combination of dormancy associated microRNAs is any one or more of DmiR1 and DmiR2; DmiR1 and DmiR3; DmiR2 and DmiR3; and DmiR1, DmiR2, and DmiR3. In other embodiments of the invention the one or more aggressiveness-associated microRNAs is any one or more of has-mir-520g, 657, and 92. In further embodiments of any of the above aspects of the invention involves the administering to the subject an effective amount of a combination of dormancy associated microRNAs.

In various embodiments of any of the above aspects or any other aspect of the invention delineated herein, the dormancy associated microRNA is expressed by a viral vector. In other embodiments the viral vector is any one or more of lentiviral vector, adenoviral vector, adeno-associated viral vector, and retroviral vector. In further embodiments the dormancy associated microRNA is delivered using a cationic liposome. In other embodiments the dormancy associated microRNA is delivered using a cationic dendrimer. In additional embodiments the dormancy associated microRNA is delivered using a nanoparticle. Other embodiments of the invention involves the step of co-administering one or more therapeutic antibodies.

In various embodiments of any of the above aspects or any other aspect of the invention delineated herein, the neoplasia is any one or more of breast carcinoma, colon carcinoma, lung carcinoma, prostate carcinoma, glioblastoma, osteosarcoma, liposarcoma, melanoma, liver carcinoma, esophageal carcinoma, and stomach carcinoma. In other embodiments the tumor is any one or more of breast carcinoma, colon carcinoma, lung carcinoma, prostate carcinoma, glioblastoma, osteosarcoma, liposarcoma, melanoma, liver carcinoma, esophageal carcinoma, and stomach carcinoma. In further embodiments the neoplastic cell is in vitro or in vivo.

In various embodiments of any of the above aspects or any other aspect of the invention delineated herein, the reference is the level of microRNA found in a dormant or fast growing tumor. In other embodiments an increase in the level of dormancy associated microRNAs indicates that the neoplasia is dormant. In further embodiments an increase in the level of aggressive associated microRNAs indicates that the neoplasia is fast-growing. In additional embodiments the reference is the level of a dormancy associated microRNA in a dormant tumor or an aggressive associated microRNA in a fast-growing tumor. In other embodiments the reference is the level of the microRNA in a sample from the subject prior to treatment or at an earlier time point during treatment.

In various embodiments of any of the above aspects or any other aspect of the invention delineated herein, an increased level of dormancy-associated microRNA indicates that conservative treatment is appropriate. In other embodiments the conservative treatment is any one or more of continued monitoring of the patient's condition, less aggressive surgery, less aggressive chemotherapy, radiotherapy, radiofrequency ablation, thermoablation via focused ultrasound, and intraartiral embolisation techniques. In additional embodiments an increased level of aggressive-associated microRNA indicates that aggressive treatment is appropriate. In further embodiments the aggressive treatment is any one or more of high dose chemotherapy, surgery, radiotherapy, radiofrequency ablation, thermoablation via focused ultrasound, and intraartiral embolisation techniques.

### DEFINITIONS

By “dormancy associated microRNA” is meant a microRNA that is expressed at a higher level in a dormant tumor than in a growing tumor. Dormancy associated microRNAs are also referred to as DmiRs.

By “DmiR1” is meant a microRNA having at least about 85% sequence identity to NCBI Accession No. NR_{—}030306 or a fragment thereof that regulates tumor dormancy, growth, survival, or proliferation. An exemplary sequence of human DmiR1 is:

| | |
|---|---|
| 1 | ataaaatttc caattggaac ctaatgattc atcagactca gatatttaag ttaacagtat |
| 61 | ttgagaatga tgaatcatta ggttccggtc agaaatt |

By “DmiR2” is meant a microRNA having at least about 85% sequence identity to NCBI Accession No. NR_{—}030316 or a fragment thereof that regulates tumor dormancy, growth, survival, or proliferation. An exemplary sequence of human DmiR2 is:

| | |
|---|---|
| 1 | agcttaggta ccaatttggc cacaatgggt tagaacacta ttccattgtg ttcttaccca |
| 61 | ccatggccaa aattgggcct aag |

By “DmiR3” is meant a microRNA having at least about 85% sequence identity to NCBI Accession No. NR_{—}029709 or a fragment thereof that regulates tumor dormancy, growth, survival, or proliferation. An exemplary sequence of human DmiR3 is:

| | |
|---|---|
| 1 | tgcaggcctc tgtgtgatat gtttgatata ttaggttgtt atttaatcca actatatatc |
| 61 | aaacatattc ctacagtgtc ttgcc |

By “has-mir 101” is meant a microRNA having at least about 85% sequence identity to NCBI Accession No. NR_{—}029516 or a fragment thereof that regulates tumor dormancy, growth, survival, or proliferation. An exemplary sequence of human has-mir 101 is:

| | |
|---|---|
| 1 | tgccctggct cagttatcac agtgctgatg ctgtctattc taaaggtaca gtactgtgat |
| 61 | aactgaagga tggca |

By “has-mir 320” is meant a microRNA having at least about 85% sequence identity to NCBI Accession No. NR_{—}029714 or a fragment thereof that regulates tumor dormancy, growth, survival, or proliferation. An exemplary sequence of human has-mir 320 is:

| | |
|---|---|
| 1 | gcttcgctcc cctccgcctt ctcttcccgg ttcttcccgg agtcgggaaa agctgggttg |
| 61 | agagggcgaa aaaggatgag gt |

By “has-mir 193b” is meant a microRNA having at least about 85% sequence identity to NCBI Accession No. NR_{—}030177 or a fragment thereof that regulates tumor dormancy, growth, survival, or proliferation. An exemplary sequence of human has-mir 193b is:

| | |
|---|---|
| 1 | gtggtctcag aatcggggtt ttgagggcga gatgagttta tgttttatcc aactggccct |
| 61 | caaagtcccg cttttggggt cat |

By “has-mir 218” is meant a microRNA having at least about 85% sequence identity to NCBI Accession No. NR_{—}029631 or a fragment thereof that regulates tumor dormancy, growth, survival, or proliferation. An exemplary sequence of human has-mir 218 is:

| | |
|---|---|
| 1 | gtgataatgt agcgagattt tctgttgtgc ttgatctaac catgtggttg cgaggtatga |
| 61 | gtaaaacatg gttccgtcaa gcaccatgga acgtcacgca gctttctaca |

By “has-mir 151” is meant a microRNA having at least about 85% sequence identity to NCBI Accession No. NR_{—}029892 or a fragment thereof that regulates tumor dormancy, growth, survival, or proliferation. An exemplary sequence of human has-mir 151 is:

| | |
|---|---|
| 1 | tttcctgccc tcgaggagct cacagtctag tatgtctcat cccctactag actgaagctc |
| 61 | cttgaggaca gggatggtca tactcacctc |

By “has-mir 19a” is meant a microRNA having at least about 85% sequence identity to NCBI Accession No. NR_{—}029489 or a fragment thereof that regulates tumor dormancy, growth, survival, or proliferation. An exemplary sequence of human has-mir 19a is:

| | |
|---|---|
| 1 | gcagtcctct gttagttttg catagttgca ctacaagaag aatgtagttg tgcaaatcta |
| 61 | tgcaaaactg atggtggcct gc |

By “has-mir 331” is meant a microRNA having at least about 85% sequence identity to NCBI Accession No. NR_{—}029895 or a fragment thereof that regulates tumor dormancy, growth, survival, or proliferation. An exemplary sequence of human has-mir 331 is:

| | |
|---|---|
| 1 | gagtttggtt ttgtttgggt ttgttctagg tatggtccca gggatcccag atcaaaccag |
| 61 | gcccctgggc ctatcctaga accaacctaa gctc |

By “has-mir 340” is meant a microRNA having at least about 85% sequence identity to NCBI Accession No. NR_{—}029885 or a fragment thereof that regulates tumor dormancy, growth, survival, or proliferation. An exemplary sequence of human has-mir 340 is:

| | |
|---|---|
| 1 | ttgtacctgg tgtgattata aagcaatgag actgattgtc atatgtcgtt tgtgggatcc |
| 61 | gtctcagtta ctttatagcc atacctggta tctta |

By “has-mir 184” is meant a microRNA having at least about 85% sequence identity to NCBI Accession No. NR_{—}029705 or a fragment thereof that regulates tumor dormancy, growth, survival, or proliferation. An exemplary sequence of human has-mir 184 is:

| | |
|---|---|
| 1 | ccagtcacgt ccccttatca cttttccagc ccagctttgt gactgtaagt gttggacgga |
| 61 | gaactgataa gggtaggtga ttga |

By “has-mir185” is meant a microRNA having at least about 85% sequence identity to NCBI Accession No. NR_{—}029706 or a fragment thereof that regulates tumor dormancy, growth, survival, or proliferation. An exemplary sequence of human has-mir185 is:

| | |
|---|---|
| 1 | agggggcgag ggattggaga gaaaggcagt tcctgatggt cccctcccca ggggctggct |
| 61 | ttcctctggt ccttccctcc ca |

By “has-mir 186” is meant a microRNA having at least about 85% sequence identity to NCBI Accession No. NR_{—}029707 or a fragment thereof that regulates tumor dormancy, growth, survival, or proliferation. An exemplary sequence of human has-mir 186 is:

| | |
|---|---|
| 1 | tgcttgtaac tttccaaaga attctccttt tgggctttct ggttttattt taagcccaaa |
| 61 | ggtgaatttt ttgggaagtt tgagct |

By “has-mir 202” is meant a microRNA having at least about 85% sequence identity to NCBI Accession No. NR_{—}030170 or a fragment thereof that regulates tumor dormancy, growth, survival, or proliferation. An exemplary sequence of human has-mir 202 is:

| | |
|---|---|
| 1 | cgcctcagag ccgcccgccg ttcctttttc ctatgcatat acttctttga ggatctggcc |
| 61 | taaagaggta tagggcatgg gaaaacgggg cggtcgggtc ctccccagcg |

By “has-mir 545” is meant a microRNA having at least about 85% sequence identity to NCBI Accession No. NR_{—}030258 or a fragment thereof that regulates tumor dormancy, growth, survival, or proliferation. An exemplary sequence of human has-mir 545 is:

| | |
|---|---|
| 1 | cgcctcagag ccgcccgccg ttcctttttc ctatgcatat acttctttga ggatctggcc |
| 61 | taaagaggta tagggcatgg gaaaacgggg cggtcgggtc ctccccagcg |

By “AmiR1” is meant a microRNA having at least about 85% sequence identity to NCBI Accession No. NR_{—}030206 or a fragment thereof that is up-regulated in growing tumors and down-regulated in dormant tumors. An exemplary sequence of human AmiR1 is:

| | |
|---|---|
| 1 | tcccatgctg tgaccctcta gaggaagcac tttctgtttg ttgtctgaga aaaaacaaag |
| 61 | tgcttccctt tagagtgtta ccgtttggga |

By “AmiR2” is meant a microRNA having at least about 85% sequence identity to NCBI Accession No. NR_{—}030394 or a fragment thereof that is up-regulated in growing tumors and down-regulated in dormant tumors. An exemplary sequence of human AmiR2 is:

| | |
|---|---|
| 1 | gtgtagtaga gctaggagga gagggtcctg gagaagcgtg gaccggtccg ggtgggttcc |
| 61 | ggcaggttct caccctctct aggccccatt ctcctctg |

By “AmiR3” is meant a microRNA having at least about 85% sequence identity to NCBI Accession No. NR_{—}029508 or a fragment thereof that is up-regulated in growing tumors and down-regulated in dormant tumors. An exemplary sequence of human AmiR3 is:

| | |
|---|---|
| 1 | ctttctacac aggttgggat cggttgcaat gctgtgtttc tgtatggtat tgcacttgtc |
| 61 | ccggcctgtt gagtttgg |

By “NFIB protein” is meant a polypeptide having 85% or greater sequence identity to a human NFIB protein and having DNA binding activity. An exemplary amino acid sequence for an NFIB protein is provided below:

| | |
|---|---|
| 1 | mmyspicltq defhpfieal lphvraiayt wfnlqarkrk yfkkhekrms kdeeravkde |
| 61 | llsekpeikq kwasrllakl rkdirqeyre dfvltvtgkk hpccvlsnpd qkgkirridc |
| 121 | lrqadkvwrl dlvmvilfkg iplestdger lmksphctnp alcvqphhit vsvkeldlfl |
| 181 | ayyvqeqdsg qsgspshndp aknppgyled sfvksgvfnv selvrvsrtp itqgtgvnfp |
| 241 | igeipsqpyy hdmnsgvnlq rslssppssk rpktisiden mepsptgdfy pspsspaags |
| 301 | rtwherdqdm sspttmkkpe kplfssaspq dssprlstfp qhhhpgipgv ahsvistrtp |
| 361 | pppsplpfpt qailppapss yfshptiryp phlnpqdtlk nyvpsydpss pqtsqpngsg |
| 421 | qvvgkvpghf tpvlapsphp savrpvtlsm tdtkpittst eaytasgtsq anryvglspr |
| 481 | dpsflhqqqs wylg |

By “NFIB polynucleotide” is meant a nucleic acid molecule encoding an NFIB protein.

By “ID 1 protein” is meant a polypeptide having at least about 85% amino acid sequence identity to an ID1 protein sequence listed in NCBI that regulates transcription. An exemplary sequence is provided at NP_{—}002156.2:

| | |
|---|---|
| 1 | mkvasgstat aaagpscalk agktasgage vvrclseqsv aisrcaggag arlpalldeq |
| 61 | qvnvllydmn gcysrlkelv ptlpqnrkvs kveilqhvid yirdlqleln sesevgtpgg |
| 121 | rglpvrapls tlngeisalt aeaacvpadd rilcr |

By “ID1 polynucleotide” is meant a nucleic acid molecule encoding an ID1 polypeptide.

By “aggressiveness-associated microRNA” is meant a microRNA that is up-regulated in aggressive or fast-growing tumors. Aggressiveness-associated microRNAs are also referred to as AmiRs.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

“Cationic dendrimer” refers to branched polymers having a positively charged surface that are used to deliver nucleic acid molecules into cells.

“Cationic lipid” refers to positively charged lipids used to deliver nucleic acid molecules into cells.

“Chemotherapeutic” means any agent useful for treating neoplasia in a subject. Chemotherapeutic includes but is not limited to abiraterone acetate, altretamine, anhydrovinblastine, auristatin, azacitidin, bendamustin, bevacizumab, bexarotene, bicalutamide, BMS184476, 2,3,4,5,6-pentafluoro-N-(3-fluoro-4-methoxyphenyl)benzene sulfonamide, bleomycin, bortezomib, N,N-dimethyl-L-valyl-L-valyl-N-methyl-L-valyl-L-proly-1-Lproline-t-butylamide, cachectin, capecitabin, cemadotin, cetuximab, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, dasatinib, daunorubicin, dolastatin, doxorubicin (adriamycin), erlotinib, etoposide, 5-fluorouracil, finasteride, flutamide, hydroxyurea and hydroxyureataxanes, ifosfamide, imatinib, irinotecan, lenalidomid, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, panitumumab, pazopanib, prednimustine, procarbazine, rituximab, RPR109881, sorafinib, stramustine phosphate, sunitinib, tamoxifen, tasonermin, taxol, temozolomide, transtuzumab, tretinoin, vinblastine, vincristine, vindesine sulfate, vinflunine, and vorinostat.

The phrase “in combination with” is intended to refer to all forms of administration that provide a DmiR molecule together with a second agent, such as a second DmiR or a chemotherapeutic agent, where the two are administered concurrently or sequentially or in any order.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control” is meant a standard or reference condition.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibits neoplasia” is meant decreases the propensity of a cell to develop into neoplasia or slows, decreases, or stabilizes the growth or proliferation of a neoplasia.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “marker” is meant any protein or polynucleotide having an alteration in expression level or activity that is associated with a disease or disorder.

By “modification” is meant any biochemical or other synthetic alteration of a nucleotide, amino acid, or other agent relative to a naturally occurring reference agent.

By “neoplasia” is meant any disease that is caused by or results in inappropriately high levels of cell division, inappropriately low levels of apoptosis, or both. For example, cancer is a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “microRNA” is meant a nucleobase sequence having biological activity that is independent of any polypeptide encoding activity. MicroRNAs may be synthetic or naturally occurring, and may include one or more modifications described herein. MicroRNAs include pri-microRNAs, hairpin microRNAs, and mature microRNAs.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

As used herein, “obtaining” as in “obtaining an agent” includes synthesizing, purchasing, or otherwise acquiring the agent.

By “oligonucleotide” is meant any molecule comprising a nucleobase sequence. An oligonucleotide may, for example, include one or more modified bases, linkages, sugar moieties, or other modifications.

The term “pharmaceutically-acceptable excipient” as used herein means one or more compatible solid or liquid filler, diluents or encapsulating substances that are suitable for administration into a human.

“Primer set” or “probe set” means a set of oligonucleotides. A primer set may be used, for example, for the amplification of a polynucleotide of interest. A probe set may be used, for example, to hybridize with a polynucleotide of interest. A primer set would consist of at least 2, 4, 6, 8, 10, 12, 14, 16, 18, 20, 30, 40, 50, 60, 80, 100, or more primers or probes.

By “reduces” is meant a negative alteration of at least 10%, 25%, 50%, 75%, or 100%.

By “reference” is meant a standard or control condition.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

“Therapeutic antibody” means any antibody or antigen-binding fragment thereof useful for treating a subject suffering from a disease.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “viral vector” refers to a virus or a fragment thereof that has been modified for the purpose of expressing a nucleic acid construct into a target cell, including but not limited to lentiviral vectors, adenoviral vectors, adeno-associated viral vector, and retroviral vector.

By “vector” is meant a nucleic acid molecule, for example, a plasmid, cosmid, or bacteriophage, that is capable of replication in a host cell. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, and enhancers.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A and 1B illustrate the isolation of Dormancy associated microRNAs (Dmirs). FIG. 1A is a schematic description of an in vivo experimental model of tumor dormancy. Briefly, human tumor cell lines injected subcutaneously in immunocompromised mice (SCID) form dormant tumors that remain undetectable by gross examination for a prolonged period of time. The dormancy period is associated with a high level of tumor cell proliferation balanced by apoptosis and impaired tumor angiogenesis which all result in restriction of tumor mass to a minimal size. Eventually, tumors spontaneously emerge from dormancy and initiate rapid tumor growth. FIG. 1B is an illustration (heat map) of the profiling results of miRNA expression levels associated with dormancy. MicroRNA expression levels in tumor cells that form dormant or fast-growing tumors from four cancer types were compared using microRNA expression cards. MicroRNAs which were differentially regulated between dormant and fast-growing tumor cells and had similar expression patterns in all cancer types analyzed are presented on the heat map.

FIGS. 2A and 2B show in vivo tumor growth rates of fast-growing glioblastoma over-expressing DmiR1, 2, or 3. FIGS. 2C and 2D illustrate the effect of DmiR over-expression on cell growth kinetics. FIG. 2A is a graph of tumor growth kinetics which were monitored in mice injected with the following cell lines: Parental fast-growing angiogenic glioblastoma cells (labeled in red line) n=11, fast-growing angiogenic glioblastoma cells infected with control GFP only viral vector (labeled in green line) n=9, fast-growing angiogenic glioblastoma cells infected with DmiR1 viral vector (labeled in black line) n=5, fast-growing angiogenic glioblastoma cells infected with DmiR2 viral vector (labeled in grey line) n=4, and fast-growing angiogenic glioblastoma cells infected with DmiR3 viral vector (labeled in blue line) n=5. FIG. 2B is a Kaplan-Meier graph of the percentage of mice with undetectable tumors over time. Tumor free mice were defined as mice bearing undetectable tumors or tumors with volumes smaller than 50 mm³ Log-rank-test values for GFP versus DmiRs expressing cells are: DmiR3: p=0.05731, DmiR1: p=0.05389 and DmiR2: p=0.08442. FIG. 2C is a graph that quantitates the in vitro proliferation of DmiR-over-expressing fast-growing glioblastoma cells. Cell proliferation was determined using an in vitro cell proliferation assay kit. The red line represents parental fast-growing glioblastoma cells, the green line represents parental fast-growing glioblastoma cells expressing GFP, the black line represents parental fast-growing glioblastoma cells expressing DmiR1, the gray line represents parental fast-growing glioblastoma cells expressing DmiR2, and the blue line represents parental fast-growing glioblastoma cells expressing DmiR3. FIG. 2D a set of photomicrographs of the immunohistochemiscal (“IHC”) analysis of fast-growing glioblastomas expressing GFP or DmiR2. A dormant tumor expressing DmiR2 (day 107 after tumor cell implantation) and a growing tumor expressing GFP (day 113 after tumor cell implantation) are presented. Tumors were stained with H&E and with an antibody for the cell proliferation marker Ki67.

FIGS. 3A and 3B are graphs illustrating the effects of DmiR over-expression on the expression levels of genes known to be associated with tumor dormancy and angiogenesis. FIG. 3A shows the relative expression levels of TIMP-3, angiomotin (Amot-1), Kras, and EphA5 in DmiR expressing clones. FIG. 3B shows the relative expression levels of TFG-alpha, HIF1a, FGF2, and EGF in microRNA expressing clones.

FIGS. 4A, 4B, and 4C demonstrate the effect of DmiR over-expression on bone marrow derived myeloid cell mobilization and recruitment. FIG. 4A is a graph of relative expression levels of Bv8 in control and DmiR over-expressing cell lines. FIG. 4B is a set of photomicrographs of GR1 and CD11b expressing cells in tumors. Fast-growing glioblastomas expressing either GFP or DmiR2 were stained for either GR1, CD11b, or CD31 markers. All tumor cells express GFP. Images of the same plane with detectors for GFP or antibody staining are shown. Scale bar represent 20 microns. Tumors were collected 73 days following injection to mice. FIG. 4C is a graph of the percentage of GR1 and CD11b expressing cells in the circulation of tumor bearing mice. The percentage of GR1 or CD11b expressing cells among CD45 positive cells in the circulation of mice bearing fast-growing glioblastoma expressing either GFP (black bars) or DmiR2 (dotted bars) was determined using fluorescence-activated cell sorting (“FACS”) analysis. Error bars represent standard error. Statistical significance was calculated using t-test. Values were p=0.0166 for differences in GR1 prevalence, p=0.0198 for CD11b prevalence and p=0.0125 for GR1/CD11b double positive cell prevalence.

FIG. 5 shows the expression levels of Dmir1, 2, and 3 in tumors obtained from human glioma patients. The expression of DmiR1 and DmiR3 was significantly decreased in advanced tumor grade in glioma specimens. WHO-I (n:3), WHO-III (n:4), and WHO-IV (n:8). P<0.01, #p=0.05.

FIG. 6 is a graph showing the effect of Dmir-3 (mir-190) over-expression on growth pattern of human osteosarcoma. Fast growing osteosarcoma cells were infected with control vector (green lines) or with mir-190 expressing vector. Each line represents one mouse. Each group had 5 mice. Note: there are 5 mice with undetectable tumors in the group that received osteosarcoma cells expressing mir-190. These tumors remained occult for over 120 days after injection.

FIG. 7 is a graph showing the effect of Dmir-3 (mir-190) over-expression on growth pattern of human liposarcoma (as tumor size over time). Fast growing liposarcoma cells were infected with control vector (green lines) or with mir-190 expressing vector. Each line represents an average of 5 tumors. There was only a modest effect for mir-190 expression on growth of fast growing liposarcoma.

FIG. 8 is a graph showing the effect of Dmir-3 (mir-190) over-expression on growth pattern of human breast carcinoma (as tumor size over time). Fast growing breast carcinoma cells were infected with control vector (green lines) or with mir-190 expressing vector. Each line represents an average of 5 tumors. There was only a modest effect for mir-190 expression on growth of fast growing breast carcinoma.

FIG. 9 is a diagram that represents a search strategy for target genes for Dmir-3 (mir-190) illustrating a comparison of bioinformatics programs for predicting binding sites for Dmir-3 (mir-190).

FIG. 10 is a table showing the eight genes that were identified as Dmir-3 target genes using a bionformatic approach comparing TargetScan, MiRanda and Pictar.

FIG. 11 is a schematic diagram that illustrates an expression profiling experiment used to identify Dmir-3 target genes in glioblastoma and osteosarcoma.

FIG. 12 is a graph showing the relative expression levels of NFIB in control or Dmir-3 (mir-190) expressing cells. The following tumor types were tested: MDA-A CT, control breast carcinoma, MDA-A-190 breast carcinoma expressing mir-190, c19 GFP, control liposarcoma and c19-190 liposarcoma expressing mir-190. Significant inhibition of NFIB is observed following Dmir-3 (mir-190) expression in both the breast carcinoma as well as the liposarcoma cells.

FIG. 13 is a graph showing the relative expression levels of TAX3b in control or Dmir-3 (mir-190) expressing cells. The following tumor types were tested: T98G A GFP control glioblastoma, T98G A 190 glioblastoma expressing mir-190, KHOS A GFP control osteosarcoma, KHOS A 190 osteosarcoma expressing mir-190, MDA-A GFP, control breast carcinoma, MDA-A-190 breast carcinoma expressing mir-190, CL9 GFP, control liposarcoma and CL 190-1 liposarcoma expressing mir-190. Significant inhibition of TAX3b is observed following Dmir-3 (mir-190) expression in all the tumor types tested.

FIG. 14 is a graph showing the relative expression levels of ID1 in control or Dmir-3 (mir-190) expressing cells. The following tumor types were: T98G A GFP control glioblastoma, T98G A 190 glioblastoma expressing mir-190, KHOS A GFP control osteosarcoma, KHOS A 190 osteosarcoma expressing mir-190, MDA-A GFP, control breast carcinoma and MDA-A-190 breast carcinoma expressing mir-190. Significant inhibition of ID1 is observed following Dmir-3 (mir-190) expression in glioblastom and breast carcinoma cells.

### DETAILED DESCRIPTION OF THE INVENTION

The invention features compositions and methods that are useful for the treatment of neoplasias (e.g. liposarcoma, breast carcinoma, glioblastoma, and osteosarcoma).

The invention is based, at least in part, on the identification of dormancy-associated microRNAs (“DmiRs”) which are uniquely up-regulated in dormant tumors, and the observation that over-expression of DmiRs in fast-growing tumor cells results in the induction of tumor dormancy. The prolongation of tumor dormancy was associated with modified profiles of gene expression and with significant reduction of GR1+/CD11b+ myeloid cells in tumors and in circulation of tumor bearing mice.

DmiRs were found by comparing the expression of microRNAs in dormant tumors to the expression of those microRNAs in fast-growing tumors (e.g., human breast carcinoma, glioblastoma, osteosarcoma, and liposarcoma tumor cells). Over 300 microRNAs were analyzed. Those microRNAs that were differentially regulated in the same pattern (i.e., those that were consistently up or down regulated) in at least three out of the four tumor types tested were selected. This provided for the identification of a consensus microRNA signature of dormant tumors, which does not depend on the type of tumor analyzed. Over-expression of either DmiR1, 2, or 3 resulted in significant prolongation of tumor dormancy in glioblastomas grown in SCID mice. Similarly, over-expression of DmiR1 or 2 resulted in inhibition of tumor growth in breast carcinoma. More microRNAs were found to be up regulated in dormant tumors than those that were down regulated. Without wishing to be bound by theory, it is likely that certain microRNAs act as tumor suppressors that inhibit or block expression of oncogenes or genes related to the escape from tumor dormancy towards rapid tumor mass expansion.

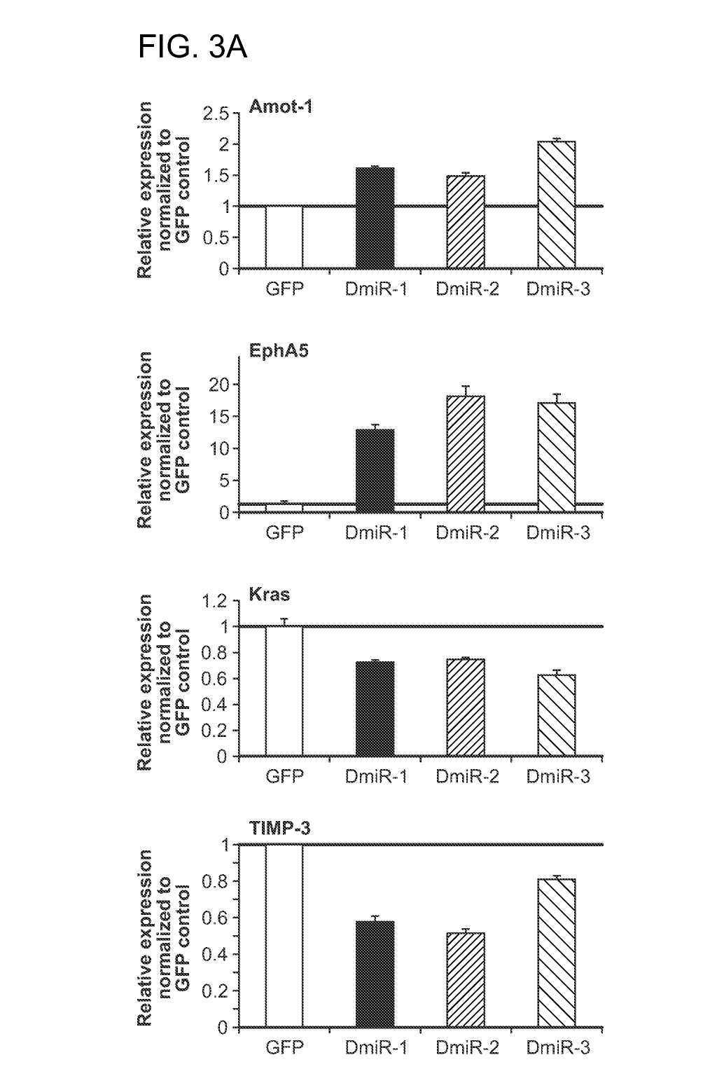

Interestingly, DmiRs expression did not affect tumor cell proliferation, but rather resulted in altered gene expression. DmiRs regulate tumor dormancy associated genes, genes involved in tumor biology and genes involved in tumor angiogenesis. Although each DmiR had a unique effect on downstream gene expression, all DmiRs tested (DmiR1, 2, and 3) had similar effects on several genes shown in FIGS. 3A and 3B. Notably, expression of EphA5, which was previously shown to be up-regulated in dormant glioblastomas (N. Almog et al., 2009), was significantly up-regulated by all DmiRs tested. Similarly, angiomotin, which is up-regulated in dormant tumors, was up-regulated following DmiR expression. TIMP-3, which is down-regulated in dormant tumors, was down-regulated by DmiRs. Therefore, these results clearly indicate a role for Dmirs as regulators of human tumor dormancy.

Tumor dormancy can result from the inability of tumor cells to induce or sustain tumor angiogenesis (J. Folkman and R. Kalluri, 2004). Although DmiRs expression resulted in upregulation of most pro-angiogenic factors tested, there was a significant decrease in expression of TGF alpha and of Bv8 in glioblastoma cells over-expressing DmiRs. Because Bv8 is a well known regulator of tissue-specific angiogenesis and haematopoietic cell mobilization (J. LeCouter et al., 2004), it prompted an investigation of the role of Bv8 in tumor dormancy.

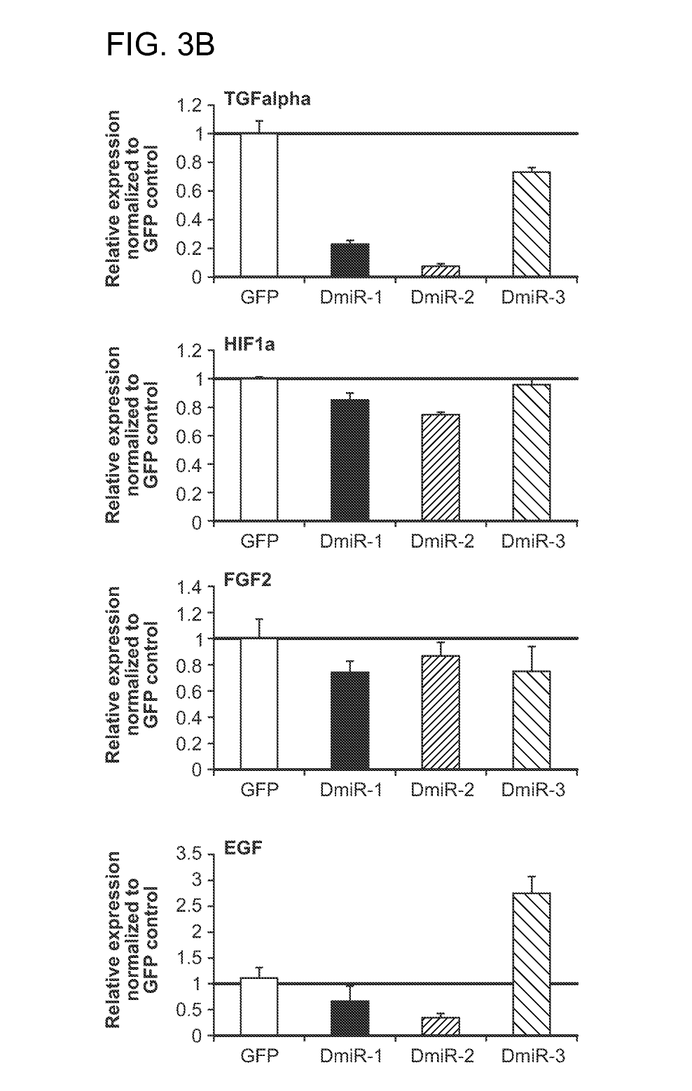

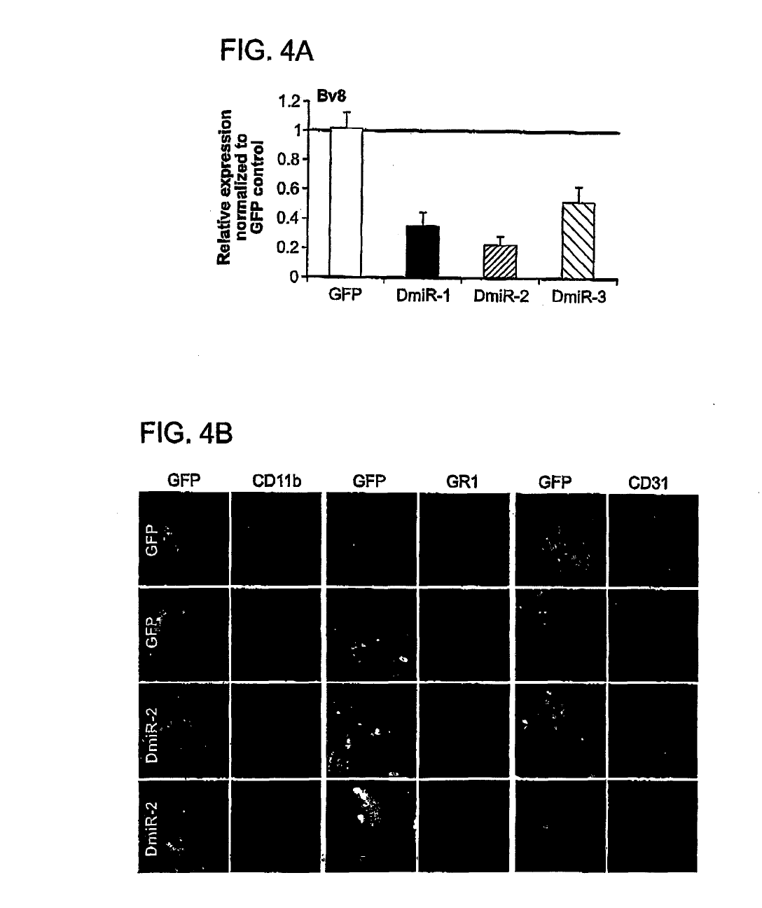

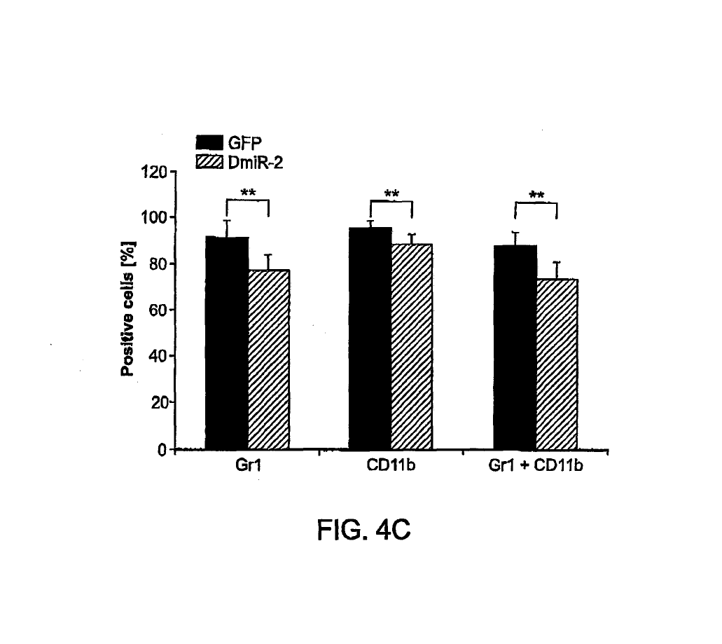

Bone marrow derived GR1+/CD11b+ myeloid cells are known to be involved in tumor growth and angiogenesis. Their mobilization and ‘tumor homing’ was shown to be associated with Bv8 (also known as prokineticin 2) expression (F. Shojaei et al., 2007). A strong reduction in prevalence of GR1+/CD11b+ myeloid cells was observed in DmiR over-expressing tumors. Moreover, while control GFP expressing glioblastomas induced mobilization of GR1+/CD11b+ myeloid cells to the circulation (FIG. 4C), levels of these cells were reduced in the circulation of mice bearing Dmir over-expressing tumors. Without wishing to be bound by theory, it is likely that the observed reduction of myeloid cells in DmiR over-expressing glioblastoma cells and in the circulation of tumor bearing mice, most likely results from the inhibition of Bv8 expression (FIG. 4A). Interestingly, granulocyte colony-stimulating factor (GCSF), the major positive regulator of Bv8, was undetectable in the glioblastoma cells tested in these assays. Without wishing to be bound by theory, it is likely that DmiR1, 2 and 3 directly effect Bv8 expression.

It is now becoming clear that many types of bone marrow-derived cells function in tumor formation and angiogenesis (Y. Shaked and E. E. Voest, 2009). It was recently reported that immature dentritic cells promote angiogenesis and that in contrast to fast growing angiogenic tumors, the dormant tumors have mostly mature dentritic cells (0. Fainaru et al., 2009). It is likely that additional bone marrow derived cells play a major role in tumor dormancy. Indeed, significant changes in levels of Lin_/Sca1+/cKit+(LSK) cells in bone marrow of mice were associated with ‘instigation’ of otherwise indolent tumors (S. S. McAllister et al., 2008). Since these cells represent the hematopoietic stem cells in mice, it is now becoming clear that several cellular populations in the bone marrow also play a role in tumor dormancy and in the transition of tumors from dormancy to growth and expansion.

The present invention provides microRNAs that can be used to block tumor progression, slow or prevent the transition of dormant tumors to fast-growing tumors, or induce dormancy in growing tumors.

### DmiR Polynucleotides

In general, the invention provides dormancy associated microRNAs (e.g., DmiR1, DmiR2, and DmiR3 as well as other tumor dormancy associated microRNA a described above). Also included in the methods of the invention are PNAs and LNAs capable of inhibiting microRNAs. An isolated nucleic acid molecule can be manipulated using recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid molecule that is isolated within a cloning or expression vector may comprise only a tiny percentage of the material in the cell in which it resides. Such a nucleic acid is isolated, however, as the term is used herein, because it can be manipulated using standard techniques known to those of ordinary skill in the art.

### DmiR Polynucleotide Therapy

As described herein, microRNAs that are expressed at higher levels in dormant tumors than in fast growing tumors (i.e., Dmirs) are useful for inhibiting, blocking, or slowing the growth or proliferation of neoplastic cells. In certain embodiments, Dmirs induce dormancy in a neoplastic cell or tumor. Accordingly, the invention provides compositions and methods for over-expressing a microRNA in a neoplastic cell or tumor. In one embodiment, a Dmir is provided directly to a neoplastic cell or tumor to induce dormancy or to otherwise inhibit tumor dormancy, growth, survival, or proliferation. In another embodiment, a vector encoding a Dmir is expressed in a neoplastic cell or tumor to induce dormancy or to otherwise inhibit tumor dormancy, growth, survival, or proliferation.

Polynucleotide therapy featuring a polynucleotide encoding a DmiR, variant, or fragment thereof is one promising therapeutic approach for treating a neoplasia. Such nucleic acid molecules can be delivered to cells of a subject having a neoplasia. The nucleic acid molecules must be delivered to the cells of a subject in a form in which they can be taken up so that therapeutically effective levels of a DmiR (e.g., DmiR1, DmiR2, and DmiR3) or fragment thereof can be produced.

Transducing viral (e.g., retroviral, lentiviral, adenoviral, and adeno-associated viral) vectors can be used for somatic cell polynucleotide therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a DmiR, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer an DmiR polynucleotide systemically.

Non-viral approaches can also be employed for the introduction of a DmiR to a cell of a patient diagnosed as having a neoplasia. For example, a Dmir or a nucleic acid molecule encoding a Dmir can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Polynucleotide transfer can also be achieved using non-viral means involving transfection in vitro. Such methods include the use of calcium phosphate, DEAE dextran, electroporation, and protoplast fusion. Liposomes can also be potentially beneficial for delivery of DNA or RNA into a cell.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant DmiR polynucleotide, variant, or fragment thereof, either directly to the site of a potential or actual disease-affected tissue or systemically (for example, by any conventional recombinant protein administration technique). The dosage of the administered polynucleotide depends on a number of factors, including the size and health of the individual patient. For any particular subject, the specific dosage regimes should be adjusted over time according to the individual need and the professional judgment of the person administering or supervising the administration of the compositions.

### Diagnostics

Dormant tumors express higher levels of dormancy associated microRNAs (e.g., DmiR1, DmiR2, and DmiR3) than corresponding fast-growing tumors. Accordingly, expression levels of differentially regulated microRNAs are correlated with a particular disease state (e.g., dormant vs. fast-growing neoplasias), and thus are useful in diagnosis. Accordingly, the present invention provides a number of diagnostic assays that are useful for the identification or characterization of a neoplasia.

In one embodiment, a patient having a neoplasia will show an alteration in the expression of a microRNA that is differentially regulated in dormant vs. fast-growing tumors. Alterations in gene expression are detected using methods known to the skilled artisan and described herein. Such information can be used to diagnose a neoplasia, to identify dormant neoplasias, or to identify fast-growing neoplasia. In another embodiment, an alteration in the expression of a DmiR is detected using real-time quantitative PCR (Q-rt-PCR).

Primers used for amplification of an DmiR molecule, including but not limited to those primer sequences described herein, are useful in diagnostic methods of the invention. The primers of the invention embrace oligonucleotides of sufficient length and appropriate sequence so as to provide specific initiation of polymerization on a significant number of nucleic acids. Specifically, the term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a locus strand. The primer must be sufficiently long to prime the synthesis of extension products in the presence of the inducing agent for polymerization. The exact length of primer will depend on many factors, including temperature, buffer, and nucleotide composition. The oligonucleotide primer typically contains between 12 and 27 or more nucleotides, although it may contain fewer nucleotides. Primers of the invention are designed to be “substantially” complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with the 5′ and 3′ flanking sequences to hybridize therewith and permit amplification of the genomic locus. While exemplary primers are provided herein, it is understood that any primer that hybridizes with the target sequences of the invention are useful in the method of the invention for detecting DmiR molecules.

In one embodiment, DmiR-specific primers amplify a desired reverse transcribed RNA target using the polymerase chain reaction (PCR). The amplified product is then detected using standard methods known in the art. In one embodiment, a PCR product (i.e., amplicon) or real-time PCR product is detected by probe binding. In one embodiment, probe binding generates a fluorescent signal, for example, by coupling a fluorogenic dye molecule and a quencher moiety to the same or different oligonucleotide substrates (e.g., TaqMan® (Applied Biosystems, Foster City, Calif., USA), Molecular Beacons (see, for example, Tyagi et al., Nature Biotechnology 14(3):303-8, 1996), Scorpions® (Molecular Probes Inc., Eugene, Oreg., USA)). In another example, a PCR product is detected by the binding of a fluorogenic dye that emits a fluorescent signal upon binding (e.g., SYBR® Green (Molecular Probes)). Such detection methods are useful for the detection of a DmiR PCR product.

In another embodiment, hybridization with PCR probes that are capable of detecting a DmiR molecule, or closely related molecules, may be used to hybridize to a nucleic acid sequence derived from a patient having a neoplasia. The specificity of the probe determines whether the probe hybridizes to a naturally occurring sequence, allelic variants, or other related sequences. Hybridization techniques may be used to monitor expression levels of these genes (for example, by Northern analysis (Ausubel et al., supra).

In general, the measurement of a DmiR molecule in a subject sample is compared with a diagnostic amount present in a reference. A diagnostic amount distinguishes between dormant tumor tissue and fast-growing tumor tissue. The skilled artisan appreciates that the particular diagnostic amount used can be adjusted to increase sensitivity or specificity of the diagnostic assay depending on the preference of the diagnostician. In general, any significant increase or decrease (e.g., at least about 30%-50%) in the level of a DmiR molecule in the subject sample relative to a reference may be used to diagnose a neoplasia, or to characterize a neoplasia as dormant or fast-growing. In one embodiment, the reference is the level of DmiR molecule present in a control sample of a corresponding dormant tumor. In another embodiment, the reference is the level of DmiR present in a corresponding tissue sample obtained from a patient that does not have a neoplasia. In another embodiment, the reference is a baseline level of DmiR present in a biologic sample derived from a patient prior to, during, or after treatment for a neoplasia. In yet another embodiment, the reference is a standardized curve.

### Types of Biological Samples

The level of a DmiR molecule can be measured in different types of biologic samples. In one embodiment, the biologic sample is a tissue sample that includes cells of a tissue or organ. Such tissue is obtained, for example, from a biopsy. In another embodiment, the biologic sample is a biologic fluid sample (e.g., blood, urine, seminal fluids, ascites, or cerebrospinal fluid.

### Kits

The invention also provides kits for the diagnosis or monitoring of a neoplasia in a biological sample obtained from a subject. In one embodiment, the kit detects an increase in the expression of a DmiR relative to a reference level of expression. In another embodiment, the kit detects an alteration in the sequence of an DmiR molecule derived from a subject relative to a reference sequence. In related embodiments, the kit includes reagents for monitoring the expression of an DmiR molecule, such as primers or probes that hybridize to a DmiR molecule.

Optionally, the kit includes directions for monitoring DmiR levels in a biological sample derived from a subject. In other embodiments, the kit comprises a sterile container which contains the primer, probe, antibody, or other detection regents; such containers can be boxes, ampoules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing a neoplasia. Preferably, the kit further comprises any one or more of the reagents described in the diagnostic assays described herein. In other embodiments, the instructions include at least one of the following: description of the primer or probe; methods for using the enclosed materials for the diagnosis of a neoplasia; precautions; warnings; indications; clinical or research studies; and/or references. The instructions may be printed directly on the container (when present), or as a label applied to the container, or as a separate sheet, pamphlet, card, or folder supplied in or with the container.

The invention also provides kits for the treatment of a neoplasia in a subject. In one embodiment, the kit includes an effective amount of a DmiR molecule and directions for using the kit for the treatment of neoplasia. In another embodiment, the kit includes an effective amount of two or more DmiR molecules.

In other embodiments, the kit comprises a sterile container which contains the DmiR molecules; such containers can be boxes, ampoules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the DmiR molecules herein and their use in treating a subject with a neoplasia. In other embodiments, the instructions include at least one of the following: methods for using the enclosed materials for the treatment of a neoplasia; precautions; warnings; indications; clinical or research studies; and/or references. The instructions may be printed directly on the container (when present), or as a label applied to the container, or as a separate sheet, pamphlet, card, or folder supplied in or with the container.

### Patient Monitoring

The disease state or treatment of a patient having a neoplasia can be monitored using the methods and compositions of the invention. In one embodiment, a probe that hybridizes to a differentially regulated microRNA is used to quantify microRNA levels, in another embodiment, a microarray is used to assay expression levels of one or more differentially regulated DmiRs. Such monitoring may be useful, for example, in assessing the efficacy of a particular drug or therapeutic regimen in a patient. In one embodiment, the expression levels of microRNAs expressed at higher levels in fast-growing tumors (Amirs) is monitored in neoplastic cells of a subject being treated for a neoplasia. In one embodiment, a reduction in the levels of such microRNAs indicates that the subject's treatment is effective, and no change in the levels of such Amirs, or an increase in the levels of Amirs indicates the subject's treatment is ineffective.

In another embodiment, the expression levels of microRNAs expressed at increased levels in Therapeutics that increase the expression of a DmiR are taken as a particularly useful in the invention.

### Screening Assays

As reported herein, the expression of a microRNAs of the invention (e.g., Dmirs and Amirs) are differentially regulated in dormant tumors vs. fast growing tumors. Agents that increase the expression of Dmirs are useful for inducing dormancy in a neoplastic cell or tumor, or for otherwise inhibiting the growth, survival, or proliferation of a neoplastic cell. Similarly, agents that reduce the expression of Amirs are useful for inducing dormancy in a neoplastic cell or tumor, or for otherwise inhibiting the growth, survival, or proliferation of a neoplastic cell. Accordingly, agents that modulate the expression or activity of a DmiR, Amir, variant, or fragment thereof are useful in the methods of the invention for the treatment or prevention of a neoplasm (e.g., breast, colon, lymph, ovary, stomach, thyroid, testis, and uterine cancer). In addition, agents that reduce the expression of an aggressiveness-associated microRNA (AmiR) whose expression is increased in a patient having a fast-growing neoplasia are also useful in the methods of the invention. Any number of methods are available for carrying out screening assays to identify agents that alter the expression of a DmiR or an AmiR. In one working example, candidate agents are added at varying concentrations to the culture medium of cultured cells expressing one of the microRNAs of the invention. MicroRNAs expression is then measured, for example, by microarray analysis, Northern blot analysis (Ausubel et al., supra), or RT-PCR, using any appropriate fragment prepared from the nucleic acid molecule as a hybridization probe. The level of microRNAexpression in the presence of the candidate agent is compared to the level measured in a control culture medium lacking the candidate molecule. An agent that promotes an alteration in the expression of the target DmiR or AmiR, or a functional equivalent thereof, is considered useful in the invention; such an agent may be used, for example, as a therapeutic to treat a neoplasia in a human patient.

In another embodiment, an expression construct is prepared whereby a detectable reporter is placed under the control of the endogenous promoter that drives DmiR or AmiR expression. The cell expressing the expression construct is then contacted with a candidate agent, and the expression of the detectable reporter in that cell is compared to the expression of the detectable reporter in an untreated control cell. A candidate compound that alters the expression of the detectable reporter is an agent that is useful for the treatment of a neoplasia. In one embodiment, the compound decreases the expression of the reporter under the control of a DmiR promoter sequence.

The invention also includes novel compounds identified by the above-described screening assays. Optionally, such compounds are characterized in one or more appropriate animal models to determine the efficacy of the compound for the treatment of a neoplasia. Desirably, characterization in an animal model can also be used to determine the toxicity, side effects, or mechanism of action of treatment with such a compound. Furthermore, novel compounds identified in any of the above-described screening assays may be used for the treatment of a neoplasia in a subject. Such agents are useful alone or in combination with other conventional therapies known in the art.

### Test Compounds and Extracts

In general, agents capable of inhibiting the growth or proliferation of a neoplasia by altering the expression or biological activity of a DmiR or AmiR, variant, or fragment thereof are identified from large libraries of either natural product or synthetic (or semi-synthetic) extracts or chemical libraries according to methods known in the art. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.).

In one embodiment, candidate agents of the invention are present in any combinatorial library known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2678-85, 1994); spatially addressable parallel solid phase or solution phase libraries; synthetic library methods requiring deconvolution; the ‘one-bead one-compound library method; and synthetic library methods using affinity chromatography selection.

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-neoplastic activity should be employed whenever possible.

Those skilled in the field of drug discovery and development will understand that the precise source of a compound or test extract is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

When a crude extract is found to alter the expression or biological activity of a Dmir, Amir, variant, or fragment thereof, further fractionation of the positive lead extract is necessary to isolate chemical constituents responsible for the observed effect. Thus, the goal of the extraction, fractionation, and purification process is the careful characterization and identification of a chemical entity within the crude extract having anti-neoplastic activity. Methods of fractionation and purification of such heterogenous extracts are known in the art. If desired, compounds shown to be useful agents for the treatment of a neoplasm are chemically modified according to methods known in the art.

### Pharmaceutical Compositions

The present invention contemplates pharmaceutical preparations comprising agents of the invention that modulate the expression of a microRNA that is differentially expressed in a dormant vs. a fast-growing tumor (e.g., a DmiR, vectors over-expressing Dmirs, an AmiR inhibitory nucleic acid molecule, a vector encoding an AmiR inhibitory nucleic acid molecule) together with a pharmaceutically acceptable carrier. Agents of the invention may be administered as part of a pharmaceutical composition. The compositions should be sterile and contain a therapeutically effective amount of the nucleic acid molecules in a unit of weight or volume suitable for administration to a subject.

These compositions ordinarily will be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous DmiR polynucleotide solution, and the resulting mixture can then be lyophilized. The infusion solution can be prepared by reconstituting the lyophilized material using sterile physiological saline solution (0.9% NaCl) or buffered solutions. In addition, DmiRs might be formulated with carrier proteins, lipids, or other organic/inorganic solutions/conjugates that facilitates tumor delivery after systemic administration. Further, alternative routes of administration, e.g. intratumoral, intrathecal, or intraaterial injections could be employed to deliver the DmiRs to target organs/tumor sites.

The agents of the invention (e.g., DmiR polynucleotide or analogs) may be combined, optionally, with a pharmaceutically acceptable excipient. The term “pharmaceutically-acceptable excipient” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with the molecules of the present invention, and with each other, in a manner such that there is no interaction that would substantially impair the desired pharmaceutical efficacy.

The compositions can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject having an neoplastic disease or disorder, an effective amount is sufficient to stabilize, slow, or reduce the proliferation of the neoplasm. Generally, doses of active polynucleotide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the compositions of the present invention (e.g., compositions comprising a DmiR).

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes. Other useful approaches are described in Otto, D. et al., J. Neurosci. Res. 22: 83-91 and in Otto, D. and Unsicker, K. J. Neurosci. 10: 1912-1921.

### Combination Therapies for the Treatment of a Neoplasm

Compositions and methods of the invention may be used in combination with any conventional therapy known in the art. In one embodiment, a composition of the invention (e.g., a composition comprising a Dmir polynucleotide) having anti-neoplastic activity may be used in combination with any anti-neoplastic therapy known in the art. Exemplary anti-neoplastic therapies include, for example, chemotherapy, cryotherapy, hormone therapy, radiotherapy, and surgery. A DmiR polynucleotide composition of the invention may, if desired, include one or more chemotherapeutics typically used in the treatment of a neoplasm, such as abiraterone acetate, altretamine, anhydrovinblastine, auristatin, azacitidin, bendamustin, bevacizumab, bexarotene, bicalutamide, BMS184476, 2,3,4,5,6-pentafluoro-N-(3-fluoro-4-methoxyphenyl)benzene sulfonamide, bleomycin, bortezomib, N,N-dimethyl-L-valyl-L-valyl-N-methyl-L-valyl-L-proly-1-Lproline-t-butylamide, cachectin, capecitabin, cemadotin, cetuximab, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, dasatinib, daunorubicin, dolastatin, doxorubicin (adriamycin), erlotinib, etoposide, 5-fluorouracil, finasteride, flutamide, hydroxyurea and hydroxyureataxanes, ifosfamide, imatinib, irinotecan, lenalidomid, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, panitumumab, pazopanib, prednimustine, procarbazine, rituximab, RPR109881, sorafinib, stramustine phosphate, sunitinib, tamoxifen, tasonermin, taxol, temozolomide, transtuzumab, tretinoin, vinblastine, vincristine, vindesine sulfate, vinflunine, and vorinostat. Other examples of chemotherapeutic agents can be found in Cancer Principles and Practice of Oncology by V. T. Devita and S. Hellman (editors), 6th edition (Feb. 15, 2001), Lippincott Williams & Wilkins Publishers.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### DmiR1, DmiR2, and DmiR3 were Significantly Up-Regulated in Dormant Tumors

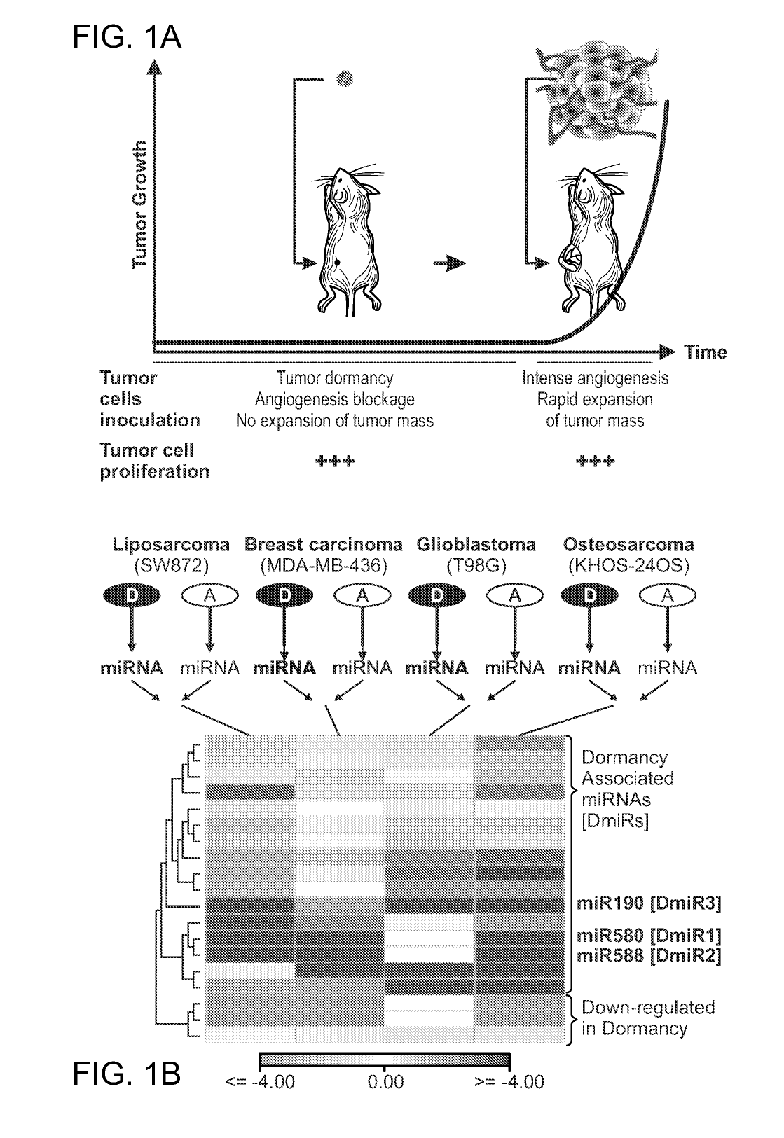

An in vivo model of human tumor dormancy comprising a panel of human tumor cell lines that form either dormant or fast-growing breast carcinoma, glioblastoma, osteosarcoma and liposarcoma was developed and used to investigate the molecular mechanisms underlying tumor dormancy. In this model, the dormant versions of the human tumor cells persisted as small (up to 2-3 mm in diameter) and asymptomatic tumors for a prolonged period of time following injection into immunodeficient mice, in contrast to the fast-growing versions (see FIG. 1A). These dormant tumors were shown to have impaired tumor angiogenesis capacity (N. Almog et al., 2006; G. N. Naumov et al., 2006). Eventually, these tumors spontaneously emerge from dormancy and undergo rapid tumor mass growth.

In order to identify microRNAs that are differentially regulated between dormant and fast-growing tumors, gene expression patterns were analyzed using total RNA harvested from both classes of cells grown in tissue. Reverse transcribed total RNA served as a substrate in quantitative PCR reactions that were set-up using pre-formed gene-specific reagent arrays (TLDA cards obtained from Applied Biosystems, CA). MicroRNA expression levels in cells that generate dormant tumors and cells that generate fast-growing angiogenic tumors were compared for each tumor type (see FIG. 1B) and microRNAs that had the same pattern of expression in all tumor types analyzed (liposarcoma, breast carcinoma, glioblastoma, and osoteosarcoma) were identified.

Ten (10) microRNAs were found to be down-regulated in dormant tumors of at least three (3) out of the four (4) tumor types analyzed. In contrast, thirty-five (35) microRNAs were found to be up-regulated in dormant tumors of at least three (3) out of the four (4) tumor types examined. The microRNAs displaying the most dramatic differential expression are shown in the heat map in FIG. 1B. MicroRNAs found to be up-regulated in dormant tumors were designated DmiRs (tumor Dormancy associated microRNAs). DmiRs shown in the heat map are has-miR-101, 320, 193b, 218, 151, 19a, 331, 340, 184, 186, 190 (DmiR3), 185, 580 (DmiR1), 588 (DmiR2), 202, and 545. MicroRNAs found to be down-regulated in dormant tumors were designated AmiRs (tumor Aggressiveness-associated microRNAs) shown on the heat map are has-miR-520g, 657 and 92.

TABLE 1
Fold differences of expression (TLDA cards data)

| | | Breast | | |
|---|---|---|---|---|
| MicroRNA | Liposarcoma | Carcinoma | Glioblastoma | Osteosarcoma |
| 193b | 1.3 | 1.4 | 1.1 | 1.8 |
| 19a | 1.8 | 1.2 | 1.5 | 1.7 |
| 101 | 1.6 | 1.3 | 1.4 | 2.5 |
| 151 | 1.4 | 1.1 | 1.2 | 1.1 |
| 184 | 2.1 | 1.2 | 2.8 | 3.9 |
| 185 | 4.3 | 2.6 | 1.1 | 2.3 |
| 186 | 1.9 | 1 | 2.4 | 2.4 |
| Dmir3 | 73.4 | 2.2 | 14.5 | 210.7 |
| 202 | 1.2 | 5.9 | 5.8 | 16.9 |
| 218 | 3.3 | 1.4 | 1.5 | 2.8 |
| 320 | 1.5 | 1.2 | 1.3 | 1.8 |
| 331 | 1.6 | 1.1 | 1.6 | 1.4 |
| 340 | 2.2 | 1.7 | 2.7 | 3.2 |
| 545 | 1.9 | 2.1 | 12.6 | 4.2 |
| DmiR1 | 8.8 | 11.7 | NA | 16.8 |
| DmiR2 | 9.8 | 12.9 | NA | 8.5 |
| 657 | 30.5 | 24 | NA | 16.1 |
| 92 | 1.2 | 1.4 | 1.5 | 1.5 |
| 520g | 15.2 | 23.4 | NA | 16.4 |

Fold difference of expression between dormant and fast growing tumors cells as obtained by real-time PCR on TLDA cards (Applied Biosystems) of the most differentially regulated microRNA. All microRNA except 657, 92 and 520g were up-regulated in dormant tumors (ratio indicates level in dormant as compared to fast growing tumors). MicroRNAs 657, 92 and 520g were down-regulated in dormant tumors (ratio indicates level in fast-growing as compared to dormant tumors). N.A indicates microRNA expression too low to be accurately determined by real-time PCR.

From the list of microRNAs displaying the most dramatic differential expression, three microRNAs (DmiR1, 2, and 3) that were significantly up-regulated in dormant tumors (see FIG. 1B) were chosen for further characterization.

The results obtained on TLDA cards was confirmed by real-time PCR analysis of individual target microRNA in each tumor type, except for the expression levels of DmiR1 and DmiR2 in glioblastoma cells (both dormant and fast-growing tumors) because their expression levels in these tumor types were below the limit of detection of real-time PCR. Both DmiR1 and DmiR3 were significantly up-regulated in cells that generate dormant tumors in all tumor types. As seen with TLDA cards, levels of DmiR2 in cells that generate dormant and fast-growing glioblastoma were too low to be detected by RT-PCR. However, DmiR2 levels in all other cell lines were too low to be accurately determined by RT-PCR as well.

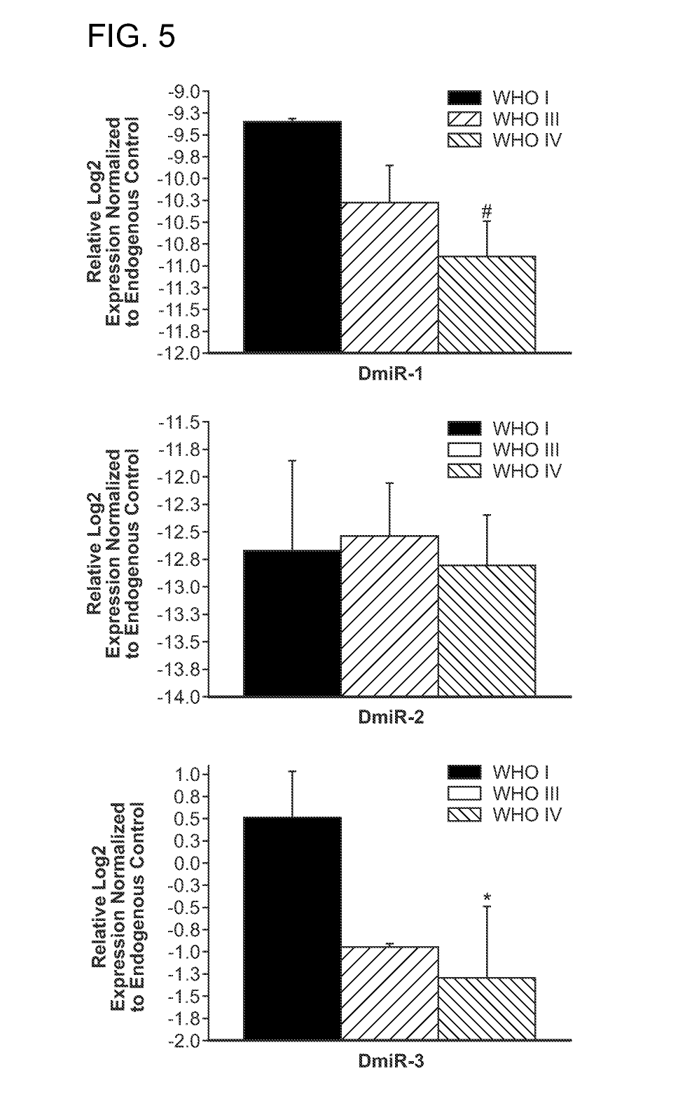

The expression levels of DmiR1, 2, and 3 was detected in human glioma specimens (FIG. 5). In support of the dormancy tumor model data, the expression of DmiR1 and DmiR3 was significantly decreased with advanced tumor grade in glioma specimens.

### Example 2

### Over-Expression of DmiRs Resulted in Prolongation of the Dormancy Period in an In Vivo Angiogenic Glioblastoma

In order to study possible roles of DmiR1, 2, and 3 in tumor dormancy, a lentiviral expression kit was used to stably over-express these microRNA in target cells. Cells that generate fast-growing glioblastomas were infected with viruses containing sequences which encode DmiR1, 2, or 3. Stably infected clones were selected by detecting the expression of the marker protein GFP and over-expression of the target DmiRs was confirmed in each of the selected stably infected cell lines by real time PCR. As expected, infection of cells that generate fast-growing glioblastoma cells (clone A=angiogenic cells) with microRNA expressing viral vectors resulted in over-expression of target DmiRs with minimal effects on the expression of other microRNAs.

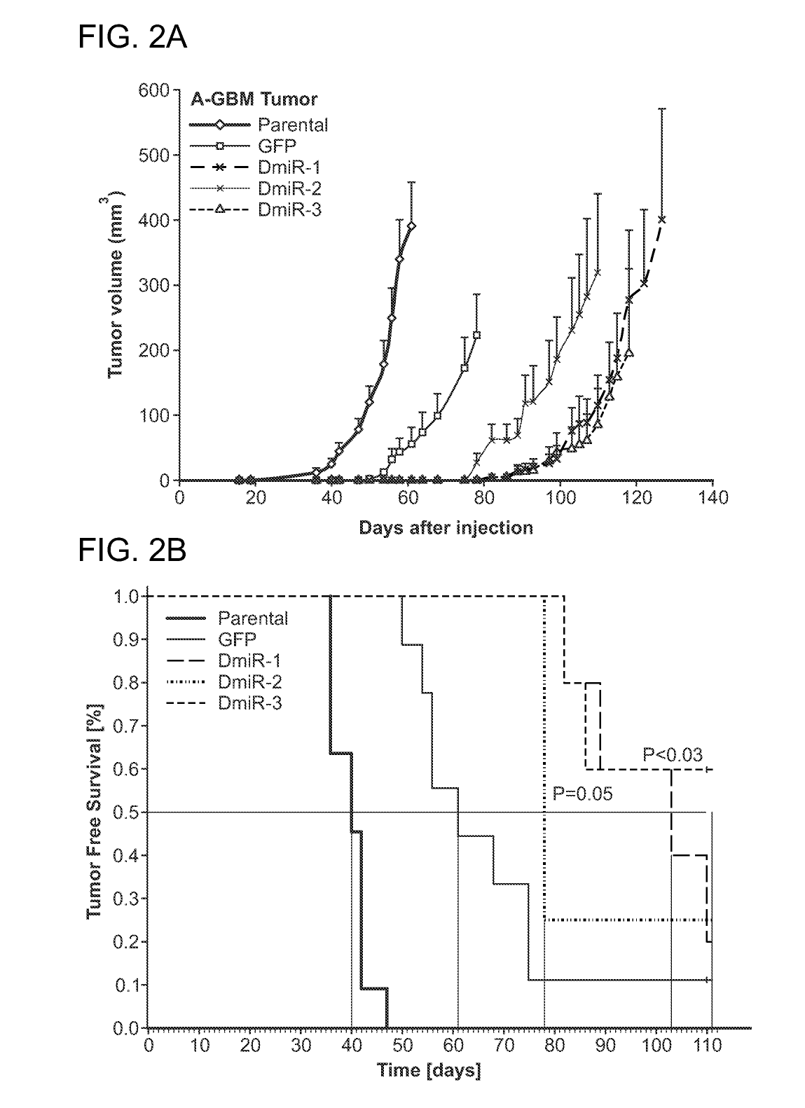

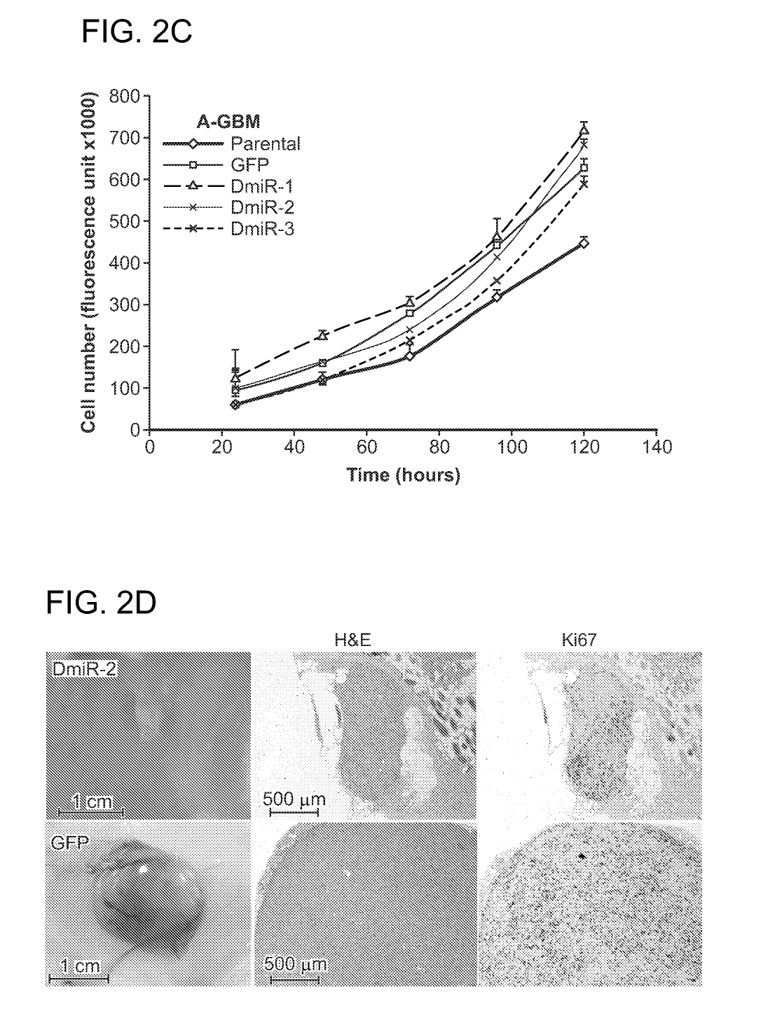

The effects of over-expression of DmiRs on the growth kinetics of the infected fast-growing angiogenic glioblastoma were determined. As expected, tumors were detected at 36-42 days following injection of each of the parental fast-growing tumor cell lines (see FIG. 2A). Infection of the tumor cells with a viral vector which express only the control GFP marker extended the dormancy period and inhibited tumor progression but not to the same extent as DmiR expression. However, the tumor development of fast-growing angiogenic glioblastomas which over-express DmiR1, 2, or 3 was significantly delayed when compared to both the parental fast-growing tumors and the control GFP expressing tumors. Over-expression of the DmiRs resulted in a longer ‘dormancy’ period which was evidenced as longer time periods in which the mice remained ‘tumor free’ or with tumors that are too small to be detected by gross examination (FIG. 2B). The “median survival” values, i.e. the time where the “survival” of “tumor free” state probability drops by 50% are significantly different: GFP by ˜60 days, DmiR3 by ˜86 days, DmiR-2 by ˜78 days, and DmiR1 by ˜103 days.

Once the DmiR expressing tumors bypass the inhibitory effect of the microRNA over-expression and initiate fast tumor growth, their growth rates are similar to those of the parental and GFP control tumors (see FIG. 2A) Although all mice with parental cell lines developed tumors (11/11), one (1) mouse out of nine (9) injected with control GFP expressing tumor cells failed to develop any detectable tumor during the experimental time period. In contrast, one (1) out of five (5) mice injected with DmiR1 over-expressing cells, one (1) out of four (4) mice injected with DmiR2 over-expressing cells, and three (3) out of five (5) mice injected with DmiR3 over-expressing cells failed to develop any detectable tumor during the experimental time period (Table 1). The most dramatic differences in tumor growth kinetics were seen between the growth patterns of GFP and of DmiR3 expressing tumors. Over-expression of DmiR3 clearly delayed tumor progression and extended the dormancy period. While all GFP expressing glioblastomas eventually ‘escaped’ from dormancy, three (3) out of five (5) DmiR3 expressing glioblastoma remained dormant for over 120 days. As observed in glioblastoma cells, over-expression of DmiR1 and DmiR2 in fast-growing breast carcinoma cells resulted in inhibition of tumor growth. Therefore, over-expression of DmiR1, 2, or 3 resulted in the prolongation of the dormancy period and postponement of the ‘switch’ from dormancy to rapid tumor mass expansion and this effect was observed in distinct tumor types.

TABLE 2
Numbers of mice with undetectable tumors

| | Undetectable | Total number of | |
|---|---|---|---|
| microRNA | tumors | mice | Percentage |
| Fast growing ‘parental’ | 0 | 11 | 0 |
| GFP (Control) | 1 | 9 | 0.11 |
| Dmir1 | 1 | 4 | 0.25 |
| Dmir2 | 1 | 5 | 0.2 |
| Dmir3 | 3 | 5 | 0.6 |

Number of mice bearing undetectable tumors or tumors with volume smaller than 50 mm³ at end point of experiment (day 64 for parental derived fast growing tumors, day 107 for control GFP expressing tumors, day 113 for Dmir2 tumors day 118 for Dmir3 tumors, and day 127 for Dmir1 tumors).

### Example 3

### Over-Expression of DmiRs Did not Effect Tumor Cell Proliferation Kinetics

The effect of DmiR over-expression on cell proliferation kinetics was determined. Cell proliferation assays revealed no differences in tumor cell proliferation kinetics in vitro among the various DmiR infected cells compared to GFP expressing control and non-infected parental control cells (FIG. 2C). Therefore, none of the DmiRs tested inhibited proliferation of tumor cells in vitro. Moreover, a high rate of cell proliferation was seen in DmiR2 expressing glioblastoma 113 days following injections of tumor cells (FIG. 2D)—a time point at which the tumor was still dormant and undetectable by gross examination but detectable only by careful examination following flipping of the mouse skin at site of injection.

### Example 4

### Over-Expression of DmiRs Altered the Expression of Known Dormancy and Tumor Related Genes

To identify the consensus target genes of DmiR1, DmiR2, and DmiR3, the expression of a panel of angiogenesis- and dormancy-related genes was profiled by quantitative RT-PCR. These genes were in part selected based on the consensus transcriptional signature that we recently reported to participate in the switch of dormant tumors to the fast-growing angiogenic phenotype. We found that anti-angiogenic and dormancy promoting genes, Angiomotin (AMOT-1) and Eph receptor A5 (EphA5) were both upregulated in DmiR1, DmiR2, and DmiR3 expressing A-GBM tumors as compared to the GFP-vector control A-GBM cells (FIG. 3A). In contrast, genes involved in pro-angiogenic signaling including tissue inhibitor of metalloproteinases 3 (TIMP-3), hypoxia-induced factor 1 alpha (HIF-1-alpha), basic fibroblast growth factor (bFGF, FGF2), and the K-ras tumor oncogene were consistently downregulated in DmiR1, DmiR2, and DmiR3 expressing A-GBM cells. Among the two epithelial growth factor receptor 1 (EGFR1) ligands, we found transforming growth factor alpha (TGT-alpha) as a common target of all three DmiRs, however epithelial growth factor was downregulated only in DmiR1 and DmiR2 expressing A-GBM cells. Importantly, we found Bv8 also known as prokineticin 2 (Prok2) to be markedly downregulated in DmiR1, DmirR2, and DmiR3 expressing A-GBM cells (FIG. 4A). Bv8 has recently discovered to play a key role in myeloid cell-dependent tumor angiogenesis and angiogenic switch. Therefore, concerted downregulation of Bv8 by all three DmiRs led us to investigate the contribution of bone marrow-derived cells (BMDCs) in the reversal of the angiogenic phenotype observed in DmiR expressing A-GBM tumors.

### Example 5

### DmiR Over-Expression Inhibited the Mobilization and Recruitment of CR1+/CD11b+ Bone Marrow Derived Myeloid Cells

Bv8 was previously shown to induce mobilization and recruitment of GR1+/CD11b+ bone marrow derived myeloid cells (J. LeCouter et al., 2004; F. Shojaei et al., 2007). The fact that Bv8 was significantly down-regulated by over-expression of DmiR1, 2, or 3 suggested that DmiR expression would result in changes in the prevalence and distribution of GR1+/CD11b+ cells; therefore, the effects of DmiR over-expression on mobilization and recruitment of these cell types was determined. Size-matched tumors over-expressing GFP (control) or DmiR2 were harvested 35 days following injection into mice and stained for GR1, Cd11b, or CD31 (FIG. 4B). Two representative control tumors are shown on two upper panels of FIG. 4B, while two representative tumors over-expressing DmiR2 are shown on lower panels of FIG. 4B. While CD11b and GR1 cells were highly abundant in control tumors, their levels were reduced in DmiR2 tumors. This reduction was more significant for GR1 cells, which were almost completely absent from DmiR2 tumors, while they were clearly present in GFP control tumors. CD31 staining, which detects tumor vasculature, was also reduced in DmiR2 tumors, suggesting reduced angiogenic capabilities of DmiR2 tumors.

Over-expression of DmiR2 in tumors also resulted in reduced levels of GR1+/CD11b+ cells in the circulation (FIG. 4C). 73 days following injection of tumor cells, peripheral blood was collected and stained for CD45, GR1, and Cd11b. As was seen by immunostaining of tumors, the levels of GR1+ cells in the circulation were more affected by DmiR2 expression than the CD11b+ cells.

### Example 6

### DmiR Expression Correlates with Tumor Stage in Patient Derived Samples

DmiR expression was examined in a panel of human gliomas surgically removed from patients. The expression levels of DmiR1, 2, and 3 were determined by real-time PCR analysis of RNA derived from fresh-frozen surgical samples. The tumor panel encompassed WHO I, WHO II, and WHO III grade tumors. The expression of the DmiRs was found to increase with increasing tumor grade.

### Example 7

### Dmir-3 Over-Expression Inhibited Tumor Growth

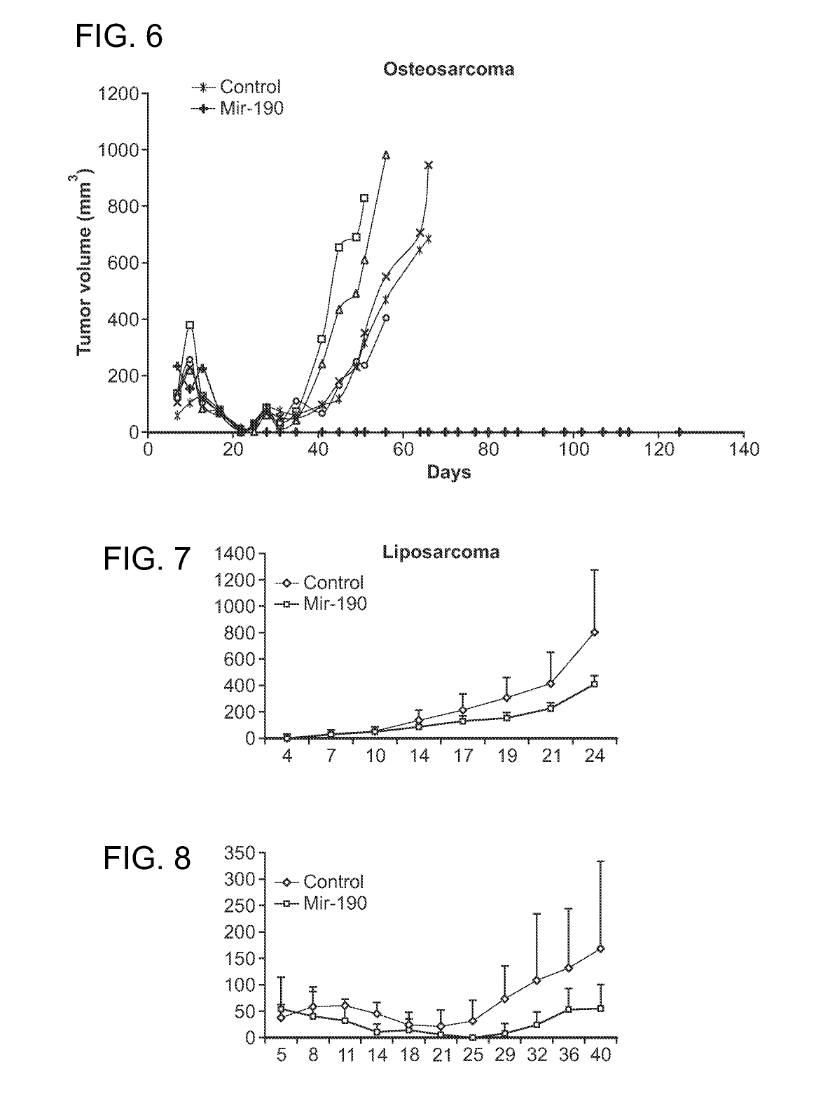

The effects of Dmir-3 over-expression were evaluated in various tumor models. Fast growing tumor cell lines were infected with vectors that over-express Dmir-3 or control vectors that lack Dmir-3. Infected cells were implanted into mice and tumor volume measured over time. Dmir-3 over-expression had a significant effect on the growth rate of osteosarcomas (FIG. 6) and liposarcomas (FIG. 7) and a modest effect on the growth rate of breast carcinomas (FIG. 8).

### Example 8

### Identification of Dmir-3 Target Genes

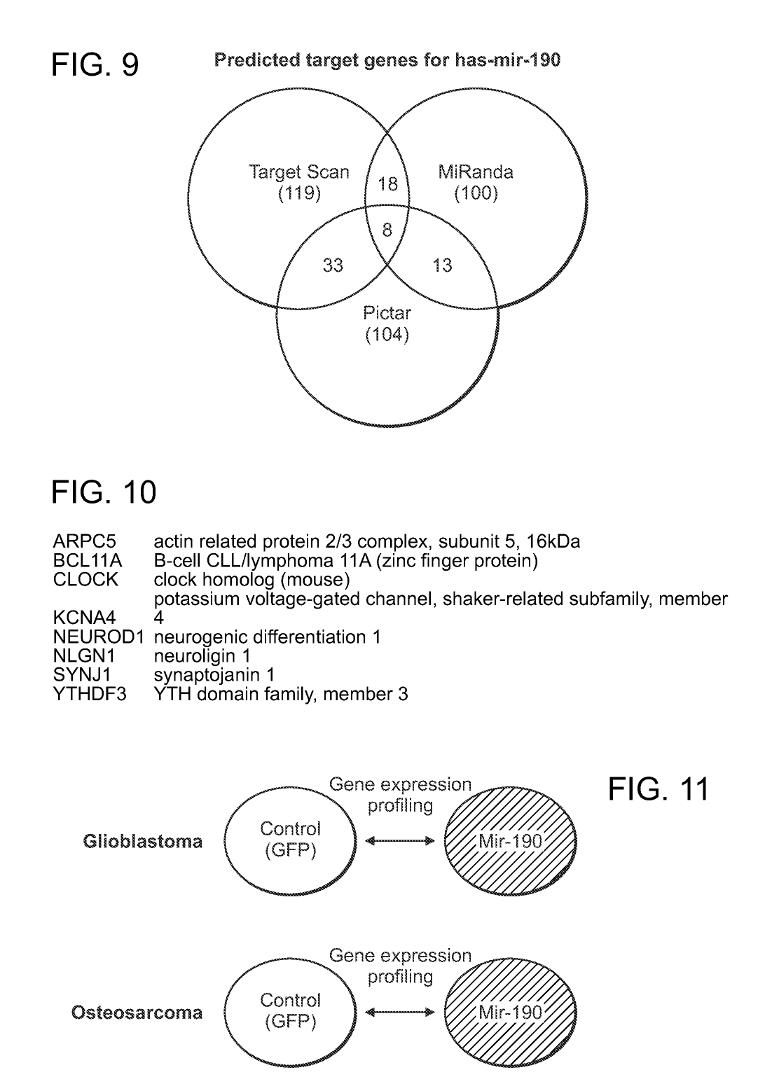

Dmir-3 target genes were identified in order to gain insight into the molecular mechanisms underlying Dmir-3's effect on tumor growth. Three bioinformatics programs were used to identify Dmir-3 target genes: TargetScan, MiRanda and Pictar. Overall results are shown in FIG. 9. Eight genes were independently identified by all three of the programs (FIG. 10).

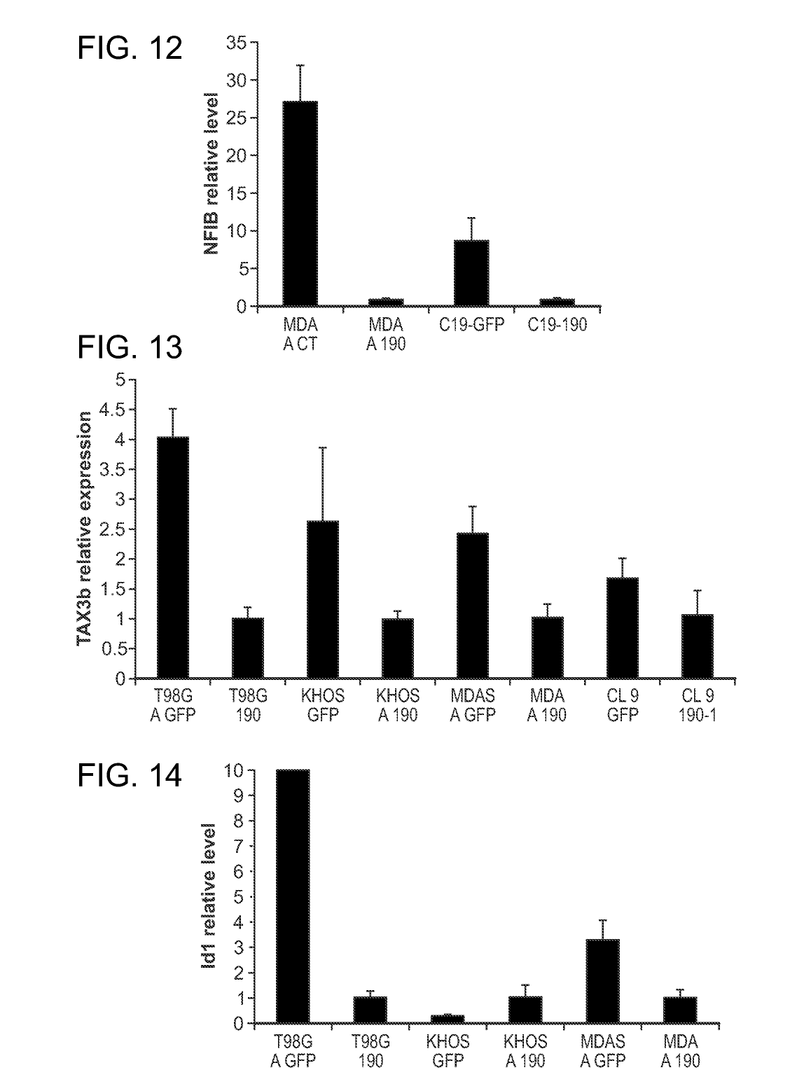

Expression profiling was also used to identify target genes that are differentially expressed in response to Dmir-3. Global expression patterns were compared between a Dmir-3 over-expressing glioblastoma and a control glioblastoma and between a Dmir-3 over-expressing osteosarcoma and a control osteosarcoma (FIG. 11). One hundred and fifty-three (153) genes were shown to be down-regulated by Dmir-3 over-expression in both glioblastoma and osteosarcoma cells. Differential expression of three of the genes (NFIB, TAX3b, TIMP-1 and ID1) was confirmed by real-time PCR in a number of cell lines: NFIB (FIG. 12); TAX3b (FIG. 13); and ID1 (FIG. 14). In particular, inhibition of ID1 was observed following mir-190 expression in glioblastom and breast carcinoma cells, and inhibition of TAX3b was observed following mir-190 expression in all the tumor types that were tested. Accordingly, the invention provides agents (e.g., small compound, polynucleotide inhibitors, antibodies) that reduce the expression or biological activity of ID1 and Tax3b.

The results described above were obtained using the following methods and materials.

### Cell Lines, Tissue Culture and Surgical Specimens

Human breast adenocarcinoma (MDA-MB-436), osteosarcoma (KHOS-24OS), glioblastoma (T98G), and liposarcoma (SW872) cell lines were obtained from the American Type Culture Collection (ATCC, Manassas, Va.). All cell lines were maintained as previously described (N. Almog et al., 2006; N. Almog et al., 2009; G. N. Naumov et al., 2006). Cell proliferation assays were performed using a cell permeable DNA-binding dye (CyQUANT NF Cell Proliferation Assay Kit obtained from Molecular Probes) according to manufacturer's instructions.

### Animals and Tumor Cell Inoculation

For in vivo experiments, sub-confluent monolayers of tumor cells were harvested by trypsinization and resuspended in DMEM to a final concentration of 2.5×10⁷ cells/mL. Male SCID mice aged 6-8 weeks (Charles River Laboratories, MA) were used for in vivo studies and were cared for in accordance with the standards of the Institutional Animal Care and Use Committee (IACUC) under a protocol approved by the Animal Care and Use Committee of St. Elizabeth Medical Center. Mice were anesthetized using a 2% isoflurane (Baxter, Deerfield, Ill.) inhalation oxygen mixture. Suspensions of 5×10⁶ human tumor cells in 0.2 mL of DMEM were then inoculated subcutaneously into the lower right quadrant of the flank of each mouse. Tumor volumes were recorded every 3-4 days. Tumor volume was calculated using the standard formula: length×width²×0.52.

### RNA Extraction and PCR

RNA was isolated using a monophasic solution of phenol and guanidine isothiocyanate (TRIzol obtained from Invitrogen) according to the manufacturer's protocol. The integrity and concentration of RNA samples was determined using RNA 6000 Nano Lab on Chip kits and an Agilent 2100 Bioanalyzer. Polymerase chain reaction (“PCR”) was performed as previously described (N. Almog et al., 2009).

### MicroRNA Expression Profiling and Analysis

Total RNA was extracted from cells grown in standard tissue culture conditions according to procedures described above. RNA was then converted into cDNA by reverse transcription (Multiplex RT for TaqMan Array Human MicroRNA Kit obtained from Applied Biosystems) and subsequently examined by gel electrophoresis to ensure viability. cDNA was then loaded on multi-well arrays containing pre-formed gene-specific quantitative PCR reagent (TLDA, TaqMan Low Density Array cards; Human MicroRNA Panel v1.0 obtained from Applied Biosystems) and microRNA expression levels were analyzed.

For Real-Time PCR analysis of microRNA, all assays were done according to the Applied Biosystems TaqMan MicroRNA Assays Protocol recommendation. Briefly, 10 ng (5 ul) of total RNA was taken per 15-ul RT reaction. Reverse transcription was performed using MultiScribe™ Reverse Transcriptase (Applied Biosystems) and the TaqMan MicroRNA Reverse Transcription Kit (Applied Biosystems). Reverse transcription reaction products were diluted 1:10 and 2 microliters were taken for PCR amplification using the TaqMan MicroRNA Assay specific to target microRNA and TqMan 2× Universal PCR Master Mix (Applied Biosystems). Assays used were TaqMan MicroRNA assay has-miR-190 (Cat #4373110,AB), TaqMan MicroRNA assay has-miR-580 (Cat #4381024,AB), TaqMan MicroRNA assay has-miR-588 (Cat #4380952,AB) TaqMan MicroRNA assay has-miR-520 (Cat #4373257,AB), TaqMan MicroRNA assay has-miR-657 (Cat #4380922,AB), and TaqMan MicroRNA assay has-miR-RNU6B was used as control (Cat #4373381,AB) where mir-580 corresponds to DmiR1, Mir-588 corresponds to DmiR2, Mir-190 corresponds to DmiR3, Mir-657 corresponds to AmiR 1, and Mir-520g corresponds to AmiR 2.

Stable Over-Expression of microRNA in Tumor Cells

Target microRNAs were introduced into tumor cells using a lentiviral vector (Lenit-miR system obtained from System Biosciences, CA.). Briefly, pre-microRNA constructs were cloned into a lentiviral vector and used together with the pPACKH1 Lentivector Packaging Kit (System Biosciences, CA.) and the 293TN Packaging Cell Line (System Biosciences, CA.) to generate infections recombinant viruses. Human tumor cells were infected with the viruses according to the manufacturer's recommendations. Stable clones were selected by expression of the marker protein GFP by FACS sorting. GFP expression was monitored throughout growth in tissue culture conditions and validated at time of tumor harvest from mice.

### Antibodies and FACS Analysis

Antibodies used for staining were purified rat anti-mouse CD11b antibody (BD Pharmingen, Franklin Lakes, N.J.) at a dilution of 1:100, purified rat anti-mouse Ly-6G and Ly-6C (anti Gr1) antibody (BD Pharmingen) at a dilution of 1:100, and purified rat anti-mouse CD31 antibody (BD Pharmingen) at a dilution of 1:100. Antibodies used for FACS analysis were all purchased from BD Pharmingen, Franklin Lakes, N.J. and include: anti-mouse CD45 (LCA, Ly-5) (BO-F11), Rat anti-mouse CD11b clone ml/70, and PE rat anti-mouse Ly-6G and Ly-6C (anti GR1 clone RB6-8C5). FACS analysis was performed using peripheral blood collected by cardiac puncture of control or tumor bearing mice. 0.9 ml blood was collected into 0.1 citrate buffer to prevent clotting. 75 μl was taken into 2 ml of Lysis buffer (RBC Lysis buffer, BioLegend), vortexed and incubated for 10 minutes at room temperature. Following centrifugation, the supernatant was collected and resuspended in FACS wash buffer (PBS, 0.1% NaN3, 1% fetal bovine serum). 1 μl of each primary antibody was added to 50 μl of blood and incubated for 30 minutes on ice. Cells were washed twice with 200 μl wash buffer and taken for FACS analysis.

### Immunofluorescence Staining of Tissue

Tumor tissues to be frozen-sectioned were dissected and slow-frozen in optimal cutting temperature compound (OCT obtained from Tissue Tek, Fisher Scientific) in the gas phase of liquid nitrogen. Frozen tumor tissues were sectioned as 15 μm sections and mounted on slides. Prior to staining, the slides were removed from −80° C. and placed at room temperature for 30 min For the CD31 antibody, the tissue sections were rinsed once in 1×PBS and were fixed by placing them in 80% methanol at −20° C. for 10 minutes and then placing them in 4% PFA for 5 minutes at room temperature. For all other antibodies, the tissue sections were rinsed once in 1×PBS and fixed in 4% PFA for 5 minutes at room temperature and washed again 3 times with 1×PBS for 5 minutes each at room temperature. To block unspecific sites the tissue sections were incubated with 1% BSA at room temperature for 1 hour followed by an additional blocking using mouse detective from Biocare Medical (Concord, Calif.) for 4 h at room temperature. The blocked sections were then washed once with 1×PBS for 5 minutes. Single antibody stains were done on tissue sections by incubating the appropriate antibody at the appropriate dilutions in 1% BSA at 4° C. overnight. The next day the tissue sections were washed with 10% goat serum 3 times for 5 minutes each at room temperature. A goat anti-rat IgG (H+L) secondary antibody (Alexa Fluor® 555 obtained from Molecular Probes, Carlsbad, Calif.) was applied to the tissue at 1:300 dilution in 10% goat serum for 1 h in dark. After applying secondary antibody, the tissue sections were washed twice in 1×PBS for 5 minutes at room temperature. For nuclear staining, 1 mM of a fluorescent nuclear dye (To-Pro-3 obtained from Molecular Probes) was diluted to 2 μM in 1×PBS and incubated on tissue sections for 15 minutes at room temperature in the dark. The tissue was rinsed once with 1×PBS for 5 minutes and anti-fade mounting solution was added. Coverslips were placed on slides, sealed with clear nail polish, and stored at −20° C.

### Microscopy

Tissue sections were viewed using a Zeiss LSM 510 Meta Confocal Scanning System (Carl Zeiss, Jena, Germany) equipped with an Argon laser, HeNe laser 543 nm, HeNe laser 633 nm, 2 one-channel detectors and one 8-channel Meta detector. Images were captured using 3 photomultiplier tubes for each detector at 1024×1024 pixels. Z-stacks were obtained when necessary. Reconstruction of z-stacks was done with Zeiss software.

### Statistical Methods

Kaplan-Meier graphs and pairwise log rank-tests were computed with a statistical software package (Statistical Utility for Microarray and Omics data, http://www.oncoexpress.org/software/sumo). The probability of an animal to convert from dormant to tumor stage was analyzed following the methodology of the Kaplan-Meier estimator. Instead of an individual's death, the time point at which tumor was first detectable by gross examination was used to build the estimator. Those animals which did not undergo this switch within the experimental period, where added as “censored” at the last time point of data acquisition (day 181).

Data are presented as mean±SEM unless otherwise noted. Statistical significance was assessed using Student's t test unless otherwise noted. P<0.05 was considered statistically significant. All statistical tests were two-tailed.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of inhibiting neoplastic cell growth, comprising contacting a neoplastic cell with a dormancy associated microRNA.

2. A method of inhibiting tumor progression, comprising contacting a tumor with an effective amount of a dormancy associated microRNA.

3. A method of prolonging dormancy in a tumor, comprising contacting the tumor with an effective amount of a dormancy associated microRNA.

4. A method of inhibiting angiogenesis in a tumor, comprising administering to the subject an effective amount of a dormancy associated microRNA.

5. A method of ameliorating a neoplasia in a subject, comprising administering to the subject an effective amount of a dormancy associated microRNA.

6. The method of any one of claims 1-5, wherein the dormancy associated microRNA is selected from the group consisting of has-miR-101, has-miR-320, has-miR-193b, has-miR-218, has-miR-151, has-miR-19a, has-miR-331, has-miR-340, has-miR-184, has-miR-186, DmiR3, has-miR-185, DmiR1, DmiR2, has-miR-202, and has-miR-545.

7. The method of any one of claims 1-5, wherein the dormancy associated microRNA is DmiR1.

8. The method of any one of claims 1-5, wherein the dormancy associated microRNA is DmiR2.

9. The method of any one of claims 1-5, wherein the dormancy associated microRNA is DmiR3.

10. The method of any one of claims 1-5, further comprising administering to the subject an effective amount of a combination of dormancy associated microRNAs.

11. The method of any one of claims 1-5, wherein the combination of dormancy associated microRNAs is selected from the group consisting of DmiR1 and DmiR2; DmiR1 and DmiR3; DmiR2 and DmiR3; and DmiR1, DmiR2, and DmiR3.

12. The method of any one of claims 1-5, wherein the dormancy associated microRNA is expressed by a viral vector.

13. The method of claim 12, wherein the viral vector is selected from the group consisting of lentiviral vector, adenoviral vector, adeno-associated viral vector, and retroviral vector.

14. The method of any one of claims 1-5, wherein the dormancy associated microRNA is delivered using a cationic liposome.

15. The method of any one of claims 1-5, wherein the dormancy associated microRNA is delivered using a cationic dendrimer.

16. The method of any one of claims 1-5, wherein the dormancy associated microRNA is delivered using a nanoparticle.

17. The method of any one of claims 1-5, further comprising the step of co-administering one or more chemotherapeutics.

18. The method of claim 17, wherein the one or more chemotherapeutics is selected from the group consisting of abiraterone acetate, altretamine, anhydrovinblastine, auristatin, azacitidin, bendamustin, bevacizumab, bexarotene, bicalutamide, BMS 184476, 2,3,4,5,6-pentafluoro-N-(3-fluoro-4-methoxyphenyl)benzene sulfonamide, bleomycin, bortezomib, N,N-dimethyl-L-valyl-L-valyl-N-methyl-L-valyl-L-proly-1-Lproline-t-butylamide, cachectin, capecitabin, cemadotin, cetuximab, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, dasatinib, daunorubicin, dolastatin, doxorubicin (adriamycin), erlotinib, etoposide, 5-fluorouracil, finasteride, flutamide, hydroxyurea and hydroxyureataxanes, ifosfamide, imatinib, irinotecan, lenalidomid, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, panitumumab, pazopanib, prednimustine, procarbazine, rituximab, RPR109881, sorafinib, stramustine phosphate, sunitinib, tamoxifen, tasonermin, taxol, temozolomide, transtuzumab, tretinoin, vinblastine, vincristine, vindesine sulfate, vinflunine, and vorinostat.

19. The method of any one of claims 1-5, further comprising the step of co-administering one or more therapeutic antibodies.

20. The method of claim 1 or 5, wherein the neoplasia is selected from the group consisting of breast carcinoma, colon carcinoma, lung carcinoma, prostate carcinoma, glioblastoma, osteosarcoma, liposarcoma, melanoma, liver carcinoma, esophageal carcinoma, and stomach carcinoma.

21. The method of any of claims 2-4, wherein the tumor is selected from the group consisting of breast carcinoma, colon carcinoma, lung carcinoma, prostate carcinoma, glioblastoma, osteosarcoma, liposarcoma, melanoma, liver carcinoma, esophageal carcinoma, and stomach carcinoma.

22. The method of claim 1 or 5 wherein the neoplastic cell is in vitro or in vivo.

23. A kit for the treatment of a neoplasia, the kit comprising an effective amount of a dormancy associated microRNA and directions for using the kit for the treatment of a neoplasia.

24. The kit of claim 23, wherein the dormancy associated microRNA is selected from the group consisting of has-miR-101, has-miR-320, has-miR-193b, has-miR-218, has-miR-151, has-miR-19a, has-miR-331, has-miR-340, has-miR-184, has-miR-186, DmiR3, has-miR-185, DmiR1, DmiR2, has-miR-202, and has-miR-545.

25. The kit of claim 23, wherein the dormancy associated microRNA is DmiR1.

26. The kit of claim 23, wherein the dormancy associated microRNA is DmiR2.

27. The kit of claim 23, wherein the dormancy associated microRNA is DmiR3.

28. The kit of claim 22, further comprising an effective amount of a combination of two or more dormancy associated microRNAs.

29. The kit of claim 27, wherein the combination of two or more dormancy associated microRNAs is selected from the group consisting of DmiR1 and DmiR2; DmiR1 and DmiR3; DmiR2 and DmiR3; and DmiR1, DmiR2, and DmiR3.

30. A pharmaceutical composition for the treatment of a neoplasia comprising an effective amount of a dormancy associated microRNA and a pharmaceutically acceptable excipient.

31. The pharmaceutical composition of claim 29, wherein the dormancy associated microRNA is selected from the group consisting of has-miR-101, has-miR-320, has-miR-193b, has-miR-218, has-miR-151, has-miR-19a, has-miR-331, has-miR-340, has-miR-184, has-miR-186, DmiR3, has-miR-185, DmiR1, DmiR2, has-miR-202, and has-miR-545.

32. The pharmaceutical composition of claim 29, wherein the dormancy associated microRNA is DmiR1.

33. The pharmaceutical composition of claim 29, wherein the dormancy associated microRNA is DmiR2.

34. The pharmaceutical composition of claim 29, wherein the dormancy associated microRNA is DmiR3.

35. The pharmaceutical composition of claim 29, further comprising an effective amount of a combination of two or more dormancy associated microRNAs.

36. The pharmaceutical composition of claim 34, wherein the combination of two or more dormancy associated microRNAs is selected from the group consisting of DmiR1 and DmiR2; DmiR1 and DmiR3; DmiR2 and DmiR3; and DmiR1, DmiR2, and DmiR3.

37. The pharmaceutical composition of claim 29, further comprising one or more chemotherapeutics.

38. The pharmaceutical composition of claim 36, wherein the one or more chemotherapeutics is selected from the group consisting of abiraterone acetate, altretamine, anhydrovinblastine, auristatin, azacitidin, bendamustin, bevacizumab, bexarotene, bicalutamide, BMS 184476, 2,3,4,5,6-pentafluoro-N-(3-fluoro-4-methoxyphenyl)benzene sulfonamide, bleomycin, bortezomib, N,N-dimethyl-L-valyl-L-valyl-N-methyl-L-valyl-L-proly-1-Lproline-t-butylamide, cachectin, capecitabin, cemadotin, cetuximab, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, dasatinib, daunorubicin, dolastatin, doxorubicin (adriamycin), erlotinib, etoposide, 5-fluorouracil, finasteride, flutamide, hydroxyurea and hydroxyureataxanes, ifosfamide, imatinib, irinotecan, lenalidomid, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, panitumumab, pazopanib, prednimustine, procarbazine, rituximab, RPR109881, sorafinib, stramustine phosphate, sunitinib, tamoxifen, tasonermin, taxol, temozolomide, transtuzumab, tretinoin, vinblastine, vincristine, vindesine sulfate, vinflunine, and vorinostat.

39. A method of characterizing the aggressiveness of a neoplasia, comprising determining the level of expression of one or more dormancy associated microRNAs in a subject sample, wherein a decreased level of expression relative to a reference indicates that the neoplasia is aggressive whereas an increased level of expression relative to a reference indicates that the neoplasia is dormant.

40. The method of claim 38, wherein the dormancy associated microRNA is selected from the group consisting of has-miR-101, has-miR-320, has-miR-193b, has-miR-218, has-miR-151, has-miR-19a, has-miR-331, has-miR-340, has-miR-184, has-miR-186, DmiR3, has-miR-185, DmiR1, DmiR2, has-miR-202, and has-miR-545.

41. A method of characterizing the aggressiveness of a neoplasia, comprising determining the level of expression of one or more aggressiveness associated microRNAs in a subject sample, wherein an increased level of expression relative to a reference indicates that the neoplasia is aggressive, whereas a decreased level of expression relative to a reference indicates that the neoplasia is dormant.

42. The method of claim 40, wherein the one or more aggressiveness-associated microRNAs is selected from the group consisting of has-mir-520g, 657, and 92.

43. The method of claim 40, wherein the reference is the level of microRNA found in a dormant or fast growing tumor.

44. A method of monitoring a subject diagnosed as having a neoplasia, the method comprising determining the level of expression of one or more dormancy associated microRNAs in a subject sample, wherein an alteration in the level of expression relative to the level of expression in a reference indicates the severity of neoplasia in a subject.

45. The method of claim 44, wherein an increase in the level of dormancy associated microRNAs indicates that the neoplasia is dormant.

46. The method of claim 44, wherein an increase in the level of aggressive associated microRNAs indicates that the neoplasia is fast-growing.

47. A method of monitoring a subject being treated for a neoplasia, the method comprising determining the level of expression of one or more aggressiveness associated microRNAs in a subject sample, wherein an alteration in the level of expression relative to the level of expression in a reference indicates the efficacy of the treatment in the subject.

48. The method of claim 44 or 47, wherein the reference is the level of a dormancy associated microRNA in a dormant tumor or an aggressive associated microRNA in a fast-growing tumor.

49. The method of claim 45, wherein the reference is the level of the microRNA in a sample from the subject prior to treatment or at an earlier time point during treatment.

50. A method of selecting a treatment regimen for a subject diagnosed as having a neoplasia, the method comprising determining the level of expression of one or more dormancy-associated or aggressive-associated microRNAs in a subject sample relative to a reference, wherein the level of expression of the microRNA indicates an appropriate treatment regimen for the subject.

51. The method of claim 50, wherein an increased level of dormancy-associated microRNA indicates that conservative treatment is appropriate.

52. The method of claim 50, wherein conservative treatment is selected from the group consisting of continued monitoring of the patient's condition, less aggressive surgery, less aggressive chemotherapy, radiotherapy, radiofrequency ablation, thermoablation via focused ultrasound, and intraartiral embolisation techniques.

53. The method of claim 50, wherein an increased level of aggressive-associated microRNA indicates that aggressive treatment is appropriate.

54. The method of claim 53, wherein aggressive treatment is selected from the group consisting of high dose chemotherapy, surgery, radiotherapy, radiofrequency ablation, thermoablation via focused ultrasound, and intraartiral embolisation techniques.

55. A diagnostic kit for the diagnosis of a neoplasia in a subject comprising a nucleic acid probe capable of detecting a dormancy-associated microRNA and written instructions for use of the kit for detection of a neoplasia.

56. A method of altering the expression of a dormancy-associated microRNA in a cell, the method comprising contacting the cell with an effective amount of an agent capable of altering the expression of the dormancy-associated microRNA.

57. A method of identifying a compound that inhibits a neoplasia, the method comprising contacting a cell that expresses a dormancy-associated microRNA with a candidate agent, and comparing the level of expression of the microRNA in the cell with the level present in a control cell not contacted by the agent, wherein an increase in expression of the dormancy-associated microRNA identifies the agent as inhibiting a neoplasia.

58. A method of identifying an agent that inhibits a neoplasia, the method comprising contacting a cell that expresses an aggressiveness-associated microRNA with a candidate compound, and comparing the level of expression of the microRNA in the cell with the level present in a control cell not contacted by the agent, wherein reduced expression of the aggressiveness-associated microRNA identifies the agent as inhibiting a neoplasia.

59. A method of identifying a candidate agent that inhibits a neoplasia, the method comprising:
a) contacting a cell containing a reporter molecule under control of a dormancy-associated microRNA promoter with a candidate compound;
b) detecting the level of the reporter molecule expressed in the cell contacted with the candidate agent; and
c) comparing the level of the reporter molecule expressed in the cell contacted with the candidate compound with the level of the reporter molecule expressed in a control cell not contacted with the candidate compound,
wherein an alteration in the level of the reporter molecule expression identifies the candidate compound as a agent that inhibits neoplasia.

60. A method of inhibiting neoplastic cell growth, comprising contacting a neoplastic cell with an agent that inhibits the expression or activity of a protein or nucleic acid molecule that is down-regulated in response to Dmir overexpression in a neoplastic cell.

61. The method of claim 60, wherein the protein is NFIB, ID1 or Tax3b, or a polynucleotide encoding said protein.

62. The method of claim 60, wherein the agent is an inhibitory nucleic acid molecule, antibody, or small compound.
